(19) United States
(12) Patent Application Publication
(10) Pub. No.: US 2019/0369106 A1
(43) Pub. Date: Dec. 5, 2019

(54) FLOW PROTEOMETRIC METHODS FOR DIGITAL QUANTIFICATION AND BINDING ANALYSIS

(71) Applicants: Board of Regents, The University of Texas System, Austin, TX (US); The Texas A&M University System, College Station, TX (US)

(72) Inventors: Mien-Chie HUNG, Houston, TX (US); Chao-Kai CHOU, Houston, TX (US); Pei-Hsiang TSOU, Taipei (TW); Po-Jung HUANG, College Station, TX (US); Jun KAMEOKA, College Station, TX (US)

(21) Appl. No.: 16/425,313

(22) Filed: May 29, 2019

Related U.S. Application Data
(60) Provisional application No. 62/677,556, filed on May 29, 2018.

(51) Int. Cl. G01N 33/58; G01N 21/64

(52) U.S. Cl. CPC G01N 33/582; G01N 2021/6441; G01N 2333/70532; G01N 21/6428

(57) ABSTRACT

Provided herein are methods and compositions for the detection and standard free quantitation of antigens of interest. Further provided are methods for the absolute quantitation of proteins in solution, cells, or tissues. Also provided are methods for determining the on-target binding constant of a therapeutic agent to its target in a biological sample.

### CROSS-REFERENCE TO RELATED APPLICATIONS

This application claims priority to U.S. Provisional Patent Application Ser. No. 62/677,556 filed May 29, 2018, the contents of which are incorporated by reference herein.

### BACKGROUND OF THE INVENTION

### 1. Field of the Invention

The present invention relates generally to the field of molecular biology. More particularly, it concerns the detection and absolute quantification of proteins from cells and tissues.

### 2. Description of Related Art

Cancer is characterized by the deregulation of signaling networks, and the identification of the deregulated molecules can support clinicians in making a precise diagnosis. A key method that allows clinicians to visualize these deregulated proteins in patient tissue samples is immunohistochemistry (IHC) (Ramos-Vara and Miller, 2014). In 2014, the U.S. Food and Drug Administration (FDA) had approved IHC-based PD-L1 level evaluation as a precursor to treat patients with the anti-PD-1 immune checkpoint therapy drug pembrolizumab (Diggs and Hsueh, 2017). PD-L1 is a transmembrane protein that is often highly expressed in tumor cells, and enable them to escape immune system attack (Chemnitz et al., 2004; Li et al., 2016). Several immune checkpoint drugs to block the PD-1/PD-L1 interactions are already approved by the FDA, directing the immune system to attack cancer cells actively (Phillips et al., 2015). While immune checkpoint therapy has successfully cured some cancer patients, the levels of PD-L1 in cancer cells can influence the treatment efficacy (Maleki Vareki et al., 2017). For instance, patients with high levels of PD-L1 expression in cancer cells present a response rate of 45%, whereas patients with low PD-L1 levels only have a response rate of 10% (Dang et al., 2016). Therefore, tumor PD-L1 level is a critical biomarker for treatment selection.

While IHC is the most commonly used method to evaluate the level of PD-L1 in the patient tumor tissues, IHC results can be biased (Matos et al., 2010). IHC bias can come from a number of sources, especially reaction bias (specimen fixation) and interpretation bias (selection and sensitivity of antibody panels) (Yaziji and Barry, 2006). Therefore, in clinical practice, it often requires multiple independent pathologists to determine an IHC result. Recently, computer-assisted imaging analyses have been developed to assist pathologists in determining the staining results (Fedchenko and Reifenrath, 2014; Jafari and Hunger, 2017). While these semi-quantitative analyses have improved the IHC results (Matos et al., 2006), it would be ideal if clinicians can have a simple way to obtain an absolute count of target protein molecules one-by-one, thus avoiding any bias inherent in the imaging analysis.

Fluorescence digital counting in a microfluidic device is a reliable method for determining biomolecule concentration. Previously, digital counting of wild-type and mutant DNA has been performed by two-color fluorescence coincidence detection in a microcapillary (Yeh et al., 2006a), and Wadhwa et al. developed a microfluidic immunoassay to detect a specific protein expressed by Mycobacterium avium subsp. paratuberculosis for distinguishing Johne's disease (Wadhwa et al., 2012). However, the common microfluidic immunoassays use the linear response between optical signal and protein concentration to estimate the analyte concentration. Therefore, in order to find out the precise analyte concentration, a comparison with a calibration standard (purified target molecules) is required. This creates a problem when the purified target protein (which serves as the standard) is difficult to obtain. In addition, these assays have a narrow dynamic range for protein concentration detection (Tekin and Gijs, 2013). Having detection sensitivity ranging from sub-nanomolar to nanomolar, fluorescence correlation spectroscopy (FCS) is another method to determine the absolute concentration of a protein by analyzing the fluorescence fluctuation in a confocal volume (˜1 μm³) (Yeh et al. 2006b), however this technique requires a long acquisition and processing times, especially when the analyte concentration is in femtomolar (fM) level or lower (Enderlein et al., 2004; Levin and Carson; 2004; Slaughter and Li, 2010). Given the need for quick turnaround times and accurate detection of target proteins in the clinic, there is an unmet need for methods to efficiently and quantitatively detect absolute protein concentration with low bias.

### SUMMARY OF THE INVENTION

In some embodiments, the present disclosure provides a method for determining the absolute concentration of an antigen of interest in a biological sample, comprising: (a) providing a sample comprising an antigen of interest; (b) contacting the biological sample with an antibody specific to the antigen of interest, wherein the antibody is conjugated to a detectable moiety; (c) optionally applying the biological sample to a microfluidic chip comprising at least one microchannel; and (d) digitally counting the detectable moiety as it passes a detection site, thereby obtaining the absolute concentration of the target protein. In some aspects, the method does not comprise the use of a calibration standard. In some aspects, the applying comprises dispensing the sample onto a microchannel and applying electroosmotic force to the sample to move the solution through the microfluidic chamber. In further aspects, the method comprises lysing the biological sample prior between steps (b) and (c). In some aspects, lysis comprises treatment with a lysis buffer. In certain aspects, the lysis buffer comprises a detergent, a salt, and a buffering agent. In particular aspects, the detergent is selected from group consisting of: non-ionic detergent, anionic detergent, cationic detergent, or zwitterionic detergent. In specific aspects, the detergent is a nonionic detergent such as NP-40, octyl-beta-glucoside, or Triton X-100. In a further aspect, lysis further comprises sonication.

In some aspects, the detection is performed using a spectrophotometer, spectroscope, or confocal microscope. In certain aspects, the detectable moiety is a fluorophore. In specific aspects, the fluorophores are selected from the group consisting of quantum dots, PE, PE-Cy5, PE-Cy7, APC, APC-Cy7, Qdot 565, qdot 605, Qdot 655, Qdot 705, green fluorescent protein (GFP), eGFP, TurboGFP, TagGFP2, mUKGEmerald GFP, Superfolder GFP, Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSS-mOrange, CyOFP1, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP657, TagRFP675, mKate2, HcRed-Tandem, mPlum, mNeptune, NirFP, Kindling, far red fluorescent protein, yellow fluorescent protein (YFP), eYFP, TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKO_{K}, mKO2, mGrapel, mGrape2, zsYellow, eqFP611, Sirius, Sandercyanin, shBFP-N158S/L173I, near infrared proteins, iFP1.4, iRFP713, iRFP670, iRFP682, iRFP702, iRFP720, iFP2.0, mIFP, TDsmURFP, miRFP670, Brilliant Violet (BV) 421, BV 605, BV 510, BV 711, BV786, PerCP, PerCP/Cy5.5, Alexa Fluor dyes such as Alexa Fluor 350, 405, 430, 488, 514, 532, 546, 555, 568, 594, 633, 635, 647, 660, 680, 700, 750, and 790, FITC, BV570, BV650, DyLight 488, Dylight 649, and PE/Dazzle 594.

In some aspects, detection further comprises optimizing the detection of the detectable moiety. In some aspects, optimizing comprises autofocusing the spectrophotometer, spectroscope, or microscope used to digitally detect the fluorophore. In some aspects, the spectrophotometer, spectroscope, or microscope is autofocused with a different wavelength laser than the laser used to excite the fluorophore. In certain aspects, the spectrophotometer, spectroscope or microscope is autofocused with a laser which excites the autofluorescence of the sample solution. In some aspects, detecting comprises Fluorescence Correlation Spectroscopy (FCS) for the measurement of flow speed. In certain aspects, digitally counting comprises detecting a photon burst by the detectable moiety as it passes through the detection site.

In some aspects of the embodiments, step (d) further comprises calculating the protein concentration of the antigen of interest from the digital count using the equation:

$$[ sample concentration ( M ) = \frac{\frac{protein events count}{6.02 \times {10}^{23}}}{detected flow volume} ] .$$

In certain aspects, the absolute detected flow volume concentration is calculated from the protein concentration using the equation:

$$[ absolute concentration ( M ) = \frac{\begin{matrix}[ sample concentration ] \times \\ absolute cell or tissue volume\end{matrix}}{sample volume} ] .$$

In some aspects of the embodiments, the samples are diluted prior to applying the sample to the microfluidic chip. In certain aspects, the samples are diluted into detection buffer. In particular aspects, the detection buffer comprises salt, EDTA, and a buffering agent. In further aspects, the detection buffer comprises glycerol. In some aspects, the time between application and detection is less than 10 minutes. In particular aspects, the time between application and detection is less than 9, 8, 7, 6, 5, 4, 3, or 2 minutes. In some aspects, the lower limit of detection in solution is 13 fM or less. In some aspects, the upper limit of detection in solution is 500 pM or less. In particular aspects, the upper limit of detection in solution is about 130 pM.

In some aspects, the biological sample comprises a plurality of cells. In certain aspects, the biological sample comprises fresh or frozen tissue. In specific aspects, the biological sample comprises formalin fixed, paraffin embedded tissue. In some aspects, the biological sample is a tissue biopsy, fine needle aspirate, blood, serum, plasma, cerebral spinal fluid, urine, stool, saliva, circulating tumor cells, exosomes, or aspirates and bodily secretions, such as sweat. In some aspects, the biological sample is fixed prior to contacting the biological sample with the antibody. In certain aspects, the sample is fixed with paraformaldehyde. In particular aspects, the sample is fixed in less than 4%, 3%, 2%, or 1% paraformaldehyde. In specific aspects, the sample is fixed in 1% paraformaldehyde. In some aspects, the sample is fixed with formaldehyde.

In some aspects, the microfluidic chip comprises two wafers. In some aspects, the microfluidic chip is between 100 and 1000 μm thick. In certain aspects, the microfluidic chip is fabricated from a polymer. In other aspects, the microfluidic chip comprises a silica wafer. In another aspect, the microfluidic chip comprises a quartz wafer. In particular aspects, the microfluidic chip comprises two quartz wafers. In certain aspects, the two quartz wafers are of different thickness. In specific aspects, the first quartz wafer is about 500 μm thick and the second quartz wafer is about 170 μm thick. In some aspects, at least one wafer has been coated with photoresist. In certain aspects, the coating of photoresist is a film of between 0.1 and 5 μm. In certain aspects, the film of photoresist is 1.5 μm thick. In particular aspects, the at least one microchannel is defined by photolithography on the photoresist. In specific aspects, the photolithography defines a pattern of two holes for paired inlet and outlet reservoirs for the at least one microchannel. In some aspects, trenches connecting the inlet and outlet reservoirs are prepared by CFH3 plasma etching, thereby generating the at least one microchannel. In specific aspects, the trenches are about 500 nm deep. In certain aspects, the microfluidic chip comprises more than 1 microchannel. In particular aspects, the microfluidic chip comprises at least 2, 3, 4, 5, 6, 7, 8, 9, 10, 11, 12, 13, 14, 15 or more microchannels. In specific aspects, the microfluidic chip comprises 14 channels. In some aspects, the two wafers are permanently fused together to close the at least one microchannel.

In aspects of the embodiments, the antigen of interest is a disease associated antigen or biomarker. In certain aspects, the antigen of interest is a cancer biomarker. In some aspects, the method further comprises detecting at least a second antigen of interest, with a at least a second antibody directed toward the second antigen of interest. In further aspects, the method is a multiplexed method.

In some embodiments, the present disclosure provides a method for determining the on-target binding constant of a therapeutic agent to its target in a biological sample comprising: (a) providing a therapeutic agent conjugated to a first detectable moiety and a biological sample comprising the target conjugated to a second detectable moiety; (b) contacting the therapeutic agent with the biological sample to bind the therapeutic agent to its target in the biological sample; (c) washing the biological sample to remove unbound therapeutic agent from the sample; (d) optionally applying the biological sample to a microfluidic chip comprising at least one microchannel; (e) digitally counting the first and second detectable moieties as they pass a detection site; and (f) calculating the on-target binding constant from the digital count of detectable moieties. In some aspects, the method further comprises lysing the biological sample prior between steps (c) and (d). In particular aspects, lysis comprises treatment with a lysis buffer. In certain aspects, the lysis buffer comprises a detergent, a salt, and a buffering agent. In some aspects, the detergent is selected from the group consisting of non-ionic detergent, anionic detergent, cationic detergent, or zwitterionic detergent. In specific aspects, the detergent is a nonionic detergent. In particular aspects, the the non-ionic detergent is NP-40, octyl-beta-glucoside, or Triton X-100. In another aspect, lysis further comprises sonication. In some aspects, the biological sample is tissue biopsy, fine needle aspirate, blood, serum, plasma, cerebral spinal fluid, urine, stool, saliva, circulating tumor cells, exosomes, or aspirates and bodily secretions, such as sweat. In some aspects, the method does not comprise the use of a calibration standard. In some aspects, the therapeutic agent is an antibody. In particular aspects, the target molecule is an antibody, disease associated antigen, ligand, virus, nucleic acid, or small molecule. In specific aspects, the target molecule is a cancer biomarker.

In some aspects, the detectable moieties are fluorophores. In certain aspects, the fluorophores of the first and second detectable moieties have different emission maxima. In particular aspects, the fluorophores of the first and second detectable moieties have different emission spectra. In specific aspects, the fluorophores of the first and second detectable moieties have non-overlapping emission spectra. In other aspects, the fluorophores of the first and second detectable moieties have overlapping emission spectra. In some aspects, the fluorophores of the first and second detectable moieties have different excitation maxima. In particular aspects, the fluorophores of the first and second detectable moieties have different excitation spectra. In specific aspects, fluorophores of the first and second detectable moieties have non-overlapping excitation spectra. In other aspects, the fluorophores of the first and second detectable moieties have overlapping excitation spectra.

In some aspects, the fluorophores are selected from the group consisting of quantum dots, PE, PE-Cy5, PE-Cy7, APC, APC-Cy7, Qdot 565, qdot 605, Qdot 655, Qdot 705, green fluorescent protein (GFP), eGFP, TurboGFP, TagGFP2, mUKG, Emerald GFP, Superfolder GFP, Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSS-mOrange, CyOFP1, Sandercyanin, red fluorescent protein (RFP), eRFP, dsRed, mRaspberry, mRuby, mApple, mCardinal, mStable, mMaroonl, mGarnet2, tdTomato, mTangerine, mStrawberry, TagRFP, TagRFP657, TagRFP675, mKate2, HcRed-Tandem, mPlum, mNeptune, NirFP, Kindling, far red fluorescent protein, yellow fluorescent protein (YFP), eYFP, TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKO_{K}, mKO2, mGrapel, mGrape2, zsYellow, eqFP611, Sirius, Sandercyanin, shBFP-N158S/L173I, near infrared proteins, iFP1.4, iRFP713, iRFP670, iRFP682, iRFP702, iRFP720, iFP2.0, mIFP, TDsmURFP, miRFP670, Brilliant Violet (BV) 421, BV 605, BV 510, BV 711, BV786, PerCP, PerCP/Cy5.5, Alexa Fluor dyes such as Alexa Fluor 350, 405, 430, 488, 514, 532, 546, 555, 568, 594, 633, 635, 647, 660, 680, 700, 750, and 790, FITC, BV570, BV650, DyLight 488, Dylight 649, and PE/Dazzle 594.

In aspects of the embodiments, digitally counting comprises detecting a photon burst from the detectable moieties as they pass through the detection site. In certain aspects, digitally counting comprises exciting the fluorophores as they pass through the detection site, and detecting a photon burst from each of the excited fluorophores. In specific aspects, on-target binding is indicated by the coincidental detection of the first and second detectable moieties. In certain aspects, the on-target binding constant is generated from coincidental detection of the first and second detectable moieties. In specific aspects, the on-target binding constant is generated using the equation:

$$B = \frac{{B}_{max} \times F}{{K}_{D} + F} .$$

In aspects of the embodiments, applying comprises dispensing the sample onto a microchannel and applying electroosmotic force to the sample to move the solution through the microfluidic chamber. In some aspects, the samples are diluted prior to step (d). In particular aspects, the samples are diluted into detection buffer. In certain aspects, the detection buffer comprises salt, EDTA, and a buffering agent. In particular aspects, the detection buffer further comprises glycerol.

In some aspects, the microfluidic chip comprises two wafers. In some aspects, the microfluidic chip is between 100 and 1000 am thick. In certain aspects, the microfluidic chip is fabricated from a polymer. In other aspects, the microfluidic chip comprises a silica wafer. In another aspect, the microfluidic chip comprises a quartz wafer. In particular aspects, the microfluidic chip comprises two quartz wafers. In certain aspects, the two quartz wafers are of different thickness. In specific aspects, the first quartz wafer is about 500 am thick and the second quartz wafer is about 170 am thick. In some aspects, at least one wafer has been coated with photoresist. In certain aspects, the coating of photoresist is a film of between 0.1 and 5 am. In certain aspects, the film of photoresist is 1.5 am thick. In particular aspects, the at least one microchannel is defined by photolithography on the photoresist. In specific aspects, the photolithography defines a pattern of two holes for paired inlet and outlet reservoirs for the at least one microchannel. In some aspects, trenches connecting the inlet and outlet reservoirs are prepared by CFH3 plasma etching, thereby generating the at least one microchannel. In specific aspects, the trenches are about 500 nm deep. In certain aspects, the microfluidic chip comprises more than 1 microchannel. In particular aspects, the microfluidic chip comprises at least 2, 3, 4, 5, 6, 7, 8, 9, 10, 11, 12, 13, 14, 15 or more microchannels. In specific aspects, the microfluidic chip comprises 14 channels. In some aspects, the two wafers are permanently fused together to close the at least one microchannel.

In some embodiments, the present disclosure provides a platform for digitally counting antigens of interest comprising a microfluidic chip, a voltage control module, an autofocus module, and a detection module. In some aspects, the detection module is a spectrophotometer, spectrometer, or confocal microscope. In some aspects, the voltage control module comprises a relay switchboard and an interface board. In certain aspects, the voltage control module controls the rate of flow thought the microfluidic chip. In specific aspects, the voltage control module controls the rate of flow by modulating electroosmotic force. In some aspects, the autofocus module regulates focus along the Z-plane. In certain aspects, regulation of focus along the Z-plane is in determined with regard to excitation of the autofluorescence of a sample solution. In specific embodiments, the wavelength used to excite the autofluorescence of the sample solution is different from the wavelength used to maximally excite a fluorophore in the sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1D: Automated single-molecule-detection platform. (a) The automated platform, which is composed of three sections: a tunable wafer holder, voltage control module, and autofocus module. (b) Optical image for the tunable wafer holder, which was built by a 3D printer. The springs on each arm are used to adjust the level of the wafer containing the microchannels. (c) The voltage control module includes a relay controller to switch on or off voltage for each microchannel; this on/off process is controlled by a computer program, which monitors the maximum intensity from each microchannel. (d) The autofocus module adjusts focusing along the z direction in order to reach the maximum intensity.

FIGS. 2A-2B: Schematic diagram for the fabrication of microchannels for detection of single molecules. (a) Top, a quartz wafer (schematic diagram) with 14 sets of inlet and outlet reservoirs connected by etched trenches, constituting 14 microchannels. Bottom, optical images of the microchannel with 200× (left) and 600× (right) magnification. (b) Summary of the microchannel fabrication steps.

FIGS. 3A-3G: Flowchart illustrating the operation of the flow-proteometric platform for analyzing protein concentration (FAP). The FAP process comprises three segments: sample preparation, sample flow through the microchannel, and data recording. Sample preparation involves (a) preparation of the sample from cells or tissue, (b) fluorescent labelling, and (c) harvest of the cell/tissue lysates. Subsequently, (d) the fluorescent-labelled samples are introduced into the microchannel; they will travel through the microchannel through electroosmosis force. (e) To enable maximum photon intensity, an autofocusing process optimizes the position of the sample before data collection. Autofocusing occurs in the x-, y-, and z-directions using a 405-nm laser. (f) A UV 635-nm laser is used to excite the fluorescent-labelled proteins. Data are recorded as the individual events (peaks) are counted digitally. (g) The protein concentration can be determined by the ratio between the counted number of events and flow volume in the focal plane during the detection time.

FIG. 4: Schematic diagram of the process of optimization. The optimization process consists of x-y alignment and z alignment. The stage moves along the x- or y-direction first, and then the microscope focus knob is rotated in order to change the focus along the z-direction. The signal readings shown here is the example readings before and after the optimization process. The optimized position reflects the maximum signal readings.

FIGS. 5A-5B: Target concentration measurement using digital counting through automated single-molecule-detection platform. (a) Photon bursts of Alexa Fluor 647-conjugated antibody with different known concentrations (0.13 to 130 pM) were detected by the automated single-molecule-detection platform. (b) Plot and the regression curve of the measured and the actual concentration. The inset shows the correlation with concentration ranged from 0.13 to 13 pM. The detection time for each measurement was 2 min. N=15. Data are mean±SEM.

FIG. 6: Measured target protein concentration using fluorescence correlation spectroscopy (FCS) for analyzing protein concentration. Alexa Fluor 647-conjugated antibody with different concentrations (0.13 to 130 pM) were detected by fluorescence correlation spectroscopy (FCS). At high concentration (100 pM), the measured concentration can precisely reflect the applied concentration, while the measured concentration could not be correlated to its applied concentration at a dilute solution (0.1 to 1 pM). The detection time for each sample is 2 min, N=15. Data represent mean±SEM.

FIGS. 7A-7C: PD-L1 expression detected by Western blot and FAP. (a) Western blot of PD-L1 detection before and after transfection of HeLa cells with green fluorescent protein (GFP)-labelled PD-L1. (b, c) Total events detected for Alexa Fluor 647-conjugated antibody against PD-L1 (specific binding) and IgG against PD-L1 (non-specific binding) in (b) HeLa PD-L1 cells and (c) MDA-MB-231 cells.

FIGS. 8A-8B: Determination of PD-L1 concentration by FAP in cultured cells and patient tissue samples. (a) PD-L1 was stably expressed in the HeLa cells. Cells were fixed labeled with Alexa Fluor 647-conjugated PD-L1 antibody. The cell lysate was collected and injected into the DCAP platform for PD-L1 detection. Control IgG was applied in the different set for measuring the nonspecific labeling (p<0.001). (b) Endogenous PD-L1 in MDA-MB-231 cells was labeled with Alexa Fluor 647-conjugated anti-PD-L antibody and similarly tested in the DCAP platform. IgG served as the negative control (p<0.001). Note that the y-axis scales differ for these graphs. The detection time for each measurement was 2 min. N=20.

FIGS. 9A-9B: Conversion of PD-L1 concentrations from cell-based samples using IgG (nonspecific binding) and PD-L1 antibody. (a) Box plot shows the converted concentrations of PD-L1 measured from HeLa cells that overexpressed PD-L; the average concentration was 357.1 nM. (b) Converted concentration of PD-L1 from MDA-MB-231 cells that overexpressed PD-L1; the average concentration was 12.3 nM. Before introducing cell or tissue lysate into the microchannel for analysis, the protein solution is processed by serial dilution in order to avoid clogging the microchannel. For cell lysates in these experiments, proteins were dissolved in lysate buffer with a final volume of 300 aL. The lysates from HeLa-PDL1 and MDA-MB-231 cells were diluted 9 times and 5 times, respectively. The methods of calculating the original PD-L1 concentration for HeLa PD-L1 and MDA-MB-231 cells are shown below.

FIGS. 10A-10B: PD-L1 concentration measurement by FAP in cancer patient tissues. (a) IHC results for PD-L1 expression in breast cancer tissue from patients A and B. (b) The measurement of PD-L1 concentration directly from patients A and B tissue using the DCAP platform (p<0.001). The detection time for each measurement was 2 min. N=20.

FIGS. 11A-11B: Converted concentrations of PD-L1 for tissue-based samples from patients with high and low levels of PD-L1 expression. (a) Photon bursts measured from the patient samples with a high (Patient A) and low (Patient B) levels of PD-L1 expression on IHC. (b) For the patient with a high level of PD-L1 expression on IHC, the FAP-measured PD-L1 concentration was 5.37 nM. For the patient with a low level of PD-L1 expression from IHC, the FAP-measured PD-L1 concentration was 0.73 nM.

FIG. 12: Measured target molecule with the concentration 13 fM through automated flow-proteometric platform. Photon bursts of Alexa Fluor 647-conjugated antibody with 13 fM (highlighted by the red circle) was detected by the automated flow-proteometric platform. The 13 fM concentration was measured under the condition EOF 400V for 8 minutes. It was plotted with other concentrations from 130 fM to 130 pM, which were measured under the condition EOF 150V for 2 minutes. For the 13 fM N=4; 130 fM to 130 pM N=15. Data are mean±SEM.

FIG. 13: Flow chart of digital quantification of antibody on-target interaction (DQON). First, cells are transfected with a target membrane protein fused with GFP; second, the cells are treated with the A647-conjugated drug; third, the cells are washed with PBS and lysed; fourth, the lysates are loaded into the microchannel for DQON analysis (also referred to as a Digital Receptor Occupancy [DRO] assay). Photon bursts from individual target proteins (green) or the drug (red) are detected; coincident photon bursts (red and green) indicate drug on-target interaction.

FIG. 14: EGFR^{GFP} and EGFRab^{A647} photon bursts detected over 5 seconds. Each red photon burst was defined as a single EGFRab^{A647} event, and each green photon burst was defined as a single EGFR^{GFP} event. A threshold was applied to exclude weak photon bursts, which might be non-specific auto-fluorescence signals. The photon bursts above the threshold were counted as events for digital quantification.

FIGS. 15A-15E: Measurement of the on-target binding KD of EGFRab in cells by event counting. (A) Representative photon bursts of EGFR^{GFP} with no EGFRab^{A647} interaction (top), EGFRabA⁶⁴⁷ with no EGFR^{GFP} interaction (middle), and EGFR^{GFP}-EGFRabA⁶⁴⁷ interaction (bottom). (B) The numbers of EGFRab^{A647}-EGFR^{GFP} events and all events involving EGFRab^{A647} after 5-60 minutes of EGFRab^{A647} treatment. (C) The ratios of EGFR^{GFP} involved in EGFRab^{A647} EGFR^{GFP} events after treatments for different times. (D) The interaction saturation curve was plotted on the basis of the ratio of EGFR^{GFP} involved in interaction with EGFRab^{A647} following treatment with different concentrations of EGFRab^{A647}. (E) The ratio of EGFRab^{A647} involved in EGFR^{GFP}-EGFRab^{A647} events. Each data point was determined based on 5,000 overall EGFR^{GFP} events counted. n=3.

FIGS. 16A-16B: Curves of EGFR^{GFP}-EGFRab^{A647} binding. (a) The overall EGFRab^{A647} events following treatment with different concentrations of EGFRab^{A647} are plotted. These EGFRab^{A647} events were associated with CHO-EGFR^{GFP} cells and were not removed by the PBS wash. (b) Only the EGFRab^{A647} events that bound with EGFR^{GFP} are plotted. Each data point was determined based on 5,000 overall EGFR^{GFP} events counted. n=3.

FIGS. 17A-17D: Quantification of the EGFRab^{A647} interaction ratio in the presence of EGF and in the tumor xenograft model. (a) CHO-EGFR^{GFP} cells were treated with or without 50 ng/ml EGF for 30 minutes at 37° C. Subsequently, the cells were treated with 15 nM EGFRab^{A647} and incubated for 5 minutes. After incubation, the cells were washed with PBS and lysed for DQON analysis. The ratio of EGFR^{GFP} involved in interaction with EGFRab^{A647} is presented. (b) Cells were treated as described for (a) but at 4° C. to suppress EGFR endocytosis. The ratio of EGFR^{GFP} involved in interaction with EGFRab^{A647} is presented. (c) Cells were treated as described for (a). The ratio of EGFRab^{A647} involved in interaction with EGFR^{GFP} presented. (d) Cells were treated as described for (a) but at 4° C. to suppress EGFR endocytosis. The ratio of EGFRab^{A647} involved in interaction with EGFR^{GFP} is presented.

FIGS. 18A-18B: Determining the ratio of EGFR that interacted with EGFRab in xenograft biopsy model. (A) Analysis of antibody on-target interaction in a xenograft model. Mice with subcutaneous GEO-EGFR^{GFP} tumors were treated with intravenous injections of 100 mg EGFRab^{A647}. Small tumor tissue samples were collected at injection and 5 and 30 minutes after injection and lysed for DQON analysis. (B) The ratios of EGFR^{GFP} targeted by EGFRab^{A647} in xenograft tumor tissue. Each data point was determined based on 5,000 overall EGFR^{GFP} events counted. n=3.

FIG. 19: Schematic diagram of the DQON optical system. Excitation laser beams (488- and 635-nm wavelengths) are focused into the center of the microchannel (2 □m wide and 500 nm deep) through a 60× water immersion objective (NA 1.2). As a single protein complex passes through the focus spot, time-coincident fluorescence emitted from the components of the complex are detected using a single-molecule spectroscope equipped with two sensitive avalanche photodiodes for observing green (GFP) and red (A647) photon signals. The system includes specific optical filters (531/40 and 685/40) to prevent signal bleed-through between different fluorophores during detection.

### DESCRIPTION OF ILLUSTRATIVE EMBODIMENTS

Immunohistochemistry has long been a key method for clinicians to detect dysregulated proteins in patient tissue samples. While IHC is the most commonly used method to evaluate the level of target antigens in the patient tumor tissues, IHC results can be biased (Matos et al., 2010). IHC bias can come from a number of sources, especially reaction bias (specimen fixation) and interpretation bias (selection and sensitivity of antibody panels) (Yaziji and Barry, 2006). Therefore, in clinical practice, it often requires multiple independent pathologists to determine an IHC result. Recently, computer-assisted imaging analyses have been developed to assist pathologists in determining the staining results, providing semi-quantitative analyses (Fedchenko and Reifenrath, 2014; Jafari and Hunger, 2017). While these semi-quantitative analyses have improved the IHC results (Matos et al., 2006), there has yet to be a simple way to get an absolute count of target protein molecules and avoid the bias inherent in IHC imaging analysis, as well as the need for purified protein standards. Provided herein are methods for determining absolute concentrations in protein molecules by digital counting.

Certain embodiments of the present disclosure provide methods and compositions for digital detection and absolute quantitation of antigens from a biological sample without the use of a standard. Methods are disclosed for the labeling of antigens and preparation of samples from cells and tissues. Also disclosed are methods for the detection and absolute quantification of labeled antigens in solution, such as by using a flow-proteometric microfluidic device. Further, methods for the production of suitable microfluidic devices for use in the detection of antigens are also disclosed. Methods are also disclosed for the measurement of the on-target binding constant of a therapeutic agent and its target, such as an antibody and its antigen.

Specifically, the present studies provide broadly applicable methods to determine the quantity of a target antigen in a cell or tissue, without the need for purified protein as a standard. The methods were used to determine the quantity of PD-L1 on a per cell basis, from both cells and tissues, including HeLa cells, cancer cells, and normal and cancerous FFPE sections. Additionally, methods provided herein were used to determine the on-target binding constant for a therapeutic agent and its target in cells and tissues. These methods were used to determine the on-target binding constant for a monoclonal EGFR antibody to EGFR in cells and resected tissues.

### I. Definitions

As used herein, the term “sample” is used in its broadest sense. A sample may include a bodily tissue or a bodily fluid including but not limited to blood (or a fraction of blood, such as plasma or serum), lymph, mucus, tears, urine, and saliva. A sample may include an extract from a cell, a chromosome, organelle, or a virus. A sample may comprise any antigen of interest. For example, a sample may comprise a protein of interest, such as an immune checkpoint inhibitor protein.

As used herein, the term “limit of detection” refers to the lowest level or amount of an antigen, such as a protein, that can be detected and quantified. Limits of detection can be represented as molar values (e.g., 130 pM limit of detection), as gram measured values (e.g., 2.0 microgram limit of detection under, for example, specified reaction conditions), copy number (e.g., 1×10⁵ copy number limit of detection), or other representations known in the art.

As used herein the term “isolated” in reference to a protein refers to a protein that is separated from the organisms and biological materials (e.g., blood, cells, serum, plasma, saliva, urine, stool, sputum, nasopharyngeal aspirates and so forth) that are present in the natural source of the nucleic acid molecule. An isolated protein, or isolated proteins, can be substantially free of other cellular material or culture medium. In some embodiments, proteins may be isolated or purified from cell lysate. Methods of protein purification and cell lysis are well known in the art.

A “label,” “imaging agent” or a “detectable moiety” is a composition detectable by spectroscopic, photochemical, biochemical, immunochemical, chemical, or other physical means. For example, useful labels include ³²P, fluorescent dyes, fluorophores, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide. Detectable moieties may be conjugated to a proteins, nucleic acids, or other target molecules to facilitate their detection.

As used herein, the term “detecting” refers to observing a signal from a detectable moiety to indicate the presence of a known target, antibody, or therapeutic agent in the sample. Any method known in the art for detecting a particular detectable moiety can be used for detection. Exemplary detection methods include, but are not limited to, spectroscopic, photochemical, biochemical, immunochemical, electrical, optical or chemical methods.

As used herein, a “fluorophore” is a chemical group that can be excited by light to emit fluorescence. Some suitable fluorophores may be excited by light to emit phosphorescence. Fluorophores have individual excitation and emission spectra. Non-limiting examples of fluorophores that may be used in the disclosed method are fluorophores such as quantum dots, PE, PE-Cy5, PE-Cy7, APC, APC-Cy7, Qdot 565, qdot 605, Qdot 655, Qdot 705, green fluorescent protein (GFP), eGFP, TurboGFP, TagGFP2, mUKGEmerald GFP, Superfolder GFP, Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSS-mOrange, CyOFP1, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP657, TagRFP675, mKate2, HcRed-Tandem, mPlum, mNeptune, NirFP, Kindling, far red fluorescent protein, yellow fluorescent protein (YFP), eYFP, TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKO_{K}, mKO2, mGrapel, mGrape2, zsYellow, eqFP611, Sirius, Sandercyanin, shBFP-N158S/L173I, near infrared proteins, iFP1.4, iRFP713, iRFP670, iRFP682, iRFP702, iRFP720, iFP2.0, mlFP, TDsmURFP, miRFP670, Brilliant Violet (BV) 421, BV 605, BV 510, BV 711, BV786, PerCP, PerCP/Cy5.5, Alexa Fluor dyes such as Alexa Fluor 350, 405, 430, 488, 514, 532, 546, 555, 568, 594, 633, 635, 647, 660, 680, 700, 750, and 790, FITC, BV570, BV650, DyLight 488, Dylight 649, and PE/Dazzle 594.

“Polypeptide,” “peptides” and “protein” are used interchangeably herein and include a molecular chain of amino acids linked through peptide bonds. The terms do not refer to a specific length of the product. The terms include post-translational modifications of the polypeptide, for example, glycosylations, acetylations, phosphorylations and the like, and also can include polypeptides that include amino acid analogs and modified peptide backbones.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “antibody” as used herein includes antibodies obtained from both polyclonal and monoclonal preparations, as well as the following: (i) hybrid (chimeric) antibody molecules (see, for example, Winter et al. (1991) Nature 349:293-299; and U.S. Pat. No. 4,816,567); (ii) F(ab′)₂ and F(ab) fragments; (iii) Fv molecules (noncovalent heterodimers, see, for example, Inbar et al. (1972) Proc. Natl. Acad. Sci. USA 69:2659-2662; and Ehrlich et al. (1980) Biochem 19:4091-4096); (iv) single-chain Fv molecules (sFv) (see, for example, Huston et al. (1988) Proc. Natl. Acad. Sci. USA 85:5879-5883); (v) dimeric and trimeric antibody fragment constructs; (vi) humanized antibody molecules (see, for example, Riechmann et al. (1988) Nature 332:323-327; Verhoeyan et al. (1988) Science 239: 1534-1536; and U. K. Patent Publication No. GB 2,276,169, published 21 Sep. 1994); (vii) Mini-antibodies or minibodies (i.e., sFv polypeptide chains that include oligomerization domains at their C-termini, separated from the sFv by a hinge region; see, e.g., Pack et al. (1992) Biochem 31:1579-1584; Cumber et al. (1992) J. Immunology 149B: 120-126); and, (vii) any functional fragments obtained from such molecules, wherein such fragments retain specific-binding properties of the parent antibody molecule.

The term “monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “polyclonal antibody” refers to a preparation of more than 1 (two or more) different antibodies to an antigen. Such a preparation includes antibodies binding to a range of different epitopes.

“Specific binding” refers to preferential binding of an antibody or therapeutic agent to a specified antigen or target relative to other non-specified antigens or targets. The phrase “specifically (or selectively) binds” to an antibody refers to a binding reaction that is determinative of the presence of the protein in a heterogeneous population of proteins and other biologics. Typically, the antibody binds with an association constant (Kₐ) of at least about 1×10⁶ M⁻¹ or 10⁷ M⁻¹, or about 108 M⁻¹ to 109 M⁻¹, or about 10¹⁰M⁻¹ to 10¹¹ M⁻¹ or higher, and binds to the specified antigen with an affinity that is at least two-fold greater than its affinity for binding to a non-specific antigen (e.g., BSA, casein) other than the specified antigen or a closely-related antigen. The phrases “an antibody recognizing an antigen” and “an antibody specific for an antigen” are used interchangeably herein with the term “an antibody which binds specifically to an antigen”. A predetermined antigen is an antigen that is chosen prior to the selection of an antibody that binds to that antigen.

The term “fixation” or “fixed” refers to increasing the mechanical strength, hardening, preserving and increasing stability of the treated biological sample such as fresh cells, biopsy or tissue, and maintains the sample in a state as similar as possible to that of the original fresh sample in situ, in its natural state. Fixation is commonly used in pathology, histology, histochemistry, cytochemistry, anatomical studies and studying cells, and generally precedes additional steps such as storage, embedding, staining, immunohistochemistry and/or immunocytochemistry. The process of fixation ideally inhibits enzymes such as nucleases and proteases, stops microbial growth on the sample and maintains both gross tissue morphology as well as cellular ultrastructure such as golgi, nucleus, endoplasmic reticulum, mitochondria, lysosomes and cytoplasmic membranes. As one example, the preservation of the correct cell morphology is important for a pathologist to diagnose the presence, type and grade of cancer in a patient, but in order to do this correctly the sample must also be capable of becoming correctly stained or labelled with antibodies for immunohistochemistry. Commonly samples are treated with a 1-5% aqueous buffered solution of formalin (formaldehyde), paraformaldehyde, or glutaraldehyde for 1-24 hours at room temperature in order to allow cross linking of proteins and other cellular components and then, following tissue sectioning, stained with Haematoxylin and Eosin stain (H&E). Although glutaraldehyde can also be used its rate of penetration into the tissue is slower than with formaldehyde (which penetrates at approximately 1 mm per hour when 18-20 volumes are added relative to the tissue volume). Whilst RNA can also be preserved in this manner, in general it becomes highly degraded during or after formalin fixation making gene expression analysis highly problematic and artifactual. One specific problem is that the RNA analyte becomes cross-linked with other biomolecules such as proteins so that they subsequently need to be released prior to analysis, this process is generally very harsh requiring extended periods at elevated temperatures which leads to significant RNA degradation. Another problem of fixation is maintaining soluble analytes such as RNA and proteins in the cell so they can be integrated by for example, in situ hybridization or immunohistochemistry. Yet another problem with formalin fixation in particular is that the covalent modification of the cellular proteins results in the loss of antigenic immunorecognition which can render immunohistochemistry techniques difficult or impossible depending on the antibody. As one further example, formalin fixed tissues are routinely embedded in paraffin wax to allow the tissue block to be thinly sliced and examined microscopically (FFPE). Other common tissue fixation methods involve using methanol, ethanol or acetone that result in protein precipitation rather than cross-linking. A review with detailed protocols has been published by Bancroft (2008) ‘Theory and Practice of Histological Techniques’ and by Stanta (2011) ‘Guidelines for Molecular Analysis in Archive Tissues’ whilst representative examples of fixed and stained tissues can be found in Ross and Pawlina (2011) ‘Histology A Text and Atlas’.

The term “treatment” or “treating” is intended to include prophylaxis, amelioration, prevention or cure of a condition (e.g., a bacterial or viral infection, or cancer). Treatment after a condition (e.g., a bacterial or viral infection, or cancer) that has started aims to reduce, ameliorate or altogether eliminate the condition, and/or its associated symptoms, or prevent it from becoming worse. Treatment of subjects before a condition has started aims to reduce the risk of developing the condition and/or lessen its severity if the condition does develop. As used herein, the term “prevent” refers to the prophylactic treatment of a subject who is at risk of developing a condition resulting in a decrease in the probability that the subject will develop the disorder, and to the inhibition of further development of an already established disorder.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is preferably below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### II. Absolute Protein Quantitation

A. Biological Sample

Certain embodiments of the present disclosure concern the detection and absolute quantification of antigens in a biological sample. As used herein, the term “biological sample” may refer to a whole organism or a subset of its tissues, cells or component parts. A “biological sample” may also refer to a homogenate, lysate, or extract prepared from a whole organism or a subset of its tissues, cells or component parts, or a fraction or portion thereof. Typically, the biological sample is diluted prior to performing an assay. Non-limiting examples of biological samples include urine, blood, cerebrospinal fluid (CSF), pleural fluid, sputum, and peritoneal fluid, bladder washings, secretions, oral washings, tissue samples, formalin fixed and paraffin embedded tissue samples, touch preps, or fine-needle aspirates. The sample may comprise body fluids and tissue samples that include but are not limited to blood, tissue biopsies, spinal fluid, meningeal fluid, urine, alveolar fluid. In some embodiments, a biological sample may be a cell line, cell culture or cell suspension. A biological sample can be from a human or non-human subject. Multiple biological samples can be characterized in a multiplexed system in which individual samples are discriminated by different detectable moieties. Further, detection of a number of different targets from a biological sample may be performed with the provided methods.

The methods of the present invention are ideally suited to characterize the presence or amount of any antigen of interest. Exemplary antigens for characterization using the methods of the present invention include programmed death protein 1 (PD-1), programmed death ligand 1 (PD-L1), programmed death ligand 2 (PD-L2), epidermal growth factor receptor (EGFR), epidermal growth factor (EGF), estrogen receptor (ER), progesterone receptor (PR), KRAS, BRAF, UGT1A1, HER2, CD4, CD8, CD25, CD20, CD30, CD38, CD134, VEGF, VEGFR, AKT, Erbl, Erb2, ErbB, Syk, JAK, JAK2, JAK3, Src, GSK3, PI3K, Ras, Raf, MAPK, MAPK1, MAPKK, mTOR, c-Kit, eph receptor, CTLA4, CABYR, CRISP3, CSAG2, CTAG2, DHFR, FOXP3, FTHL17, GAGE1, LDHC, MAGEAi, MAGEA3, MAGEA4, MAGEB6, MICA, MUC1, NLRP4, NYESO1, OX40 P53, PBK, PRAME, SOX2, SPANXAi, SSX2, SSX4, SSX5, STAT1, STAT3, STAT4, STAT5, T-bet, TSGA10, TSSK6, TULP2, XAGE2, ZNF167, CXCR3, CXCR4, CCR5, CCR4. CCR7 CCR8, CCR1, IL-113, IL-2, IL-4, IL-5, IL-6, IL-8, IL-10, IL-12, IFN-γ, TNF-αt, GM-CSF, caspase-3, other cancer and immune system biomarkers, and antibodies directed towards these antigens or other antigens of interest.

B. Detection Methods

1. Microfluidic Devices

A submicrometer fluid channel, also referred to as a nanofluidic channel or microfluidic channel, is a nanofabricated structure that physically constrains the sample in two dimensions, one lateral and one axial, reducing the number of unwanted fluorophores detected. Another property of the nanofluidic channels is the ability to flow single molecules through the detection volume with a high degree of control. This enables a balance of several factors important to single molecule detection and analysis, including detection efficiency and rates of throughput and data acquisition. While the nanofluidic channel has been described as a channel having a cross section of approximately 500 nm square, other sizes of channels having similar characteristics may be used.

The microfluidic or nanofluidic channels may be made from a variety of materials, including, but not limited to silica, quartz, glass, or polymers such as polydimethylsiloxane (PDMS). In some embodiments, the microfluidic channels are made from ultraviolet grade silica wafers. A base wafer is coated with a think layer of photoresist (e.g. photoresist S1813), which is then used to lithographically define the microchannel pattern. The microchannel pattern can then be created by reacted-ion etching. Following etching, the photoresist layer is removed, and a cover silica wafer with reservoir holes is thermally bonded to the patterned wafer to seal the microchannels. Plastic reservoirs are then glued to the holes as inlet and outlets for the samples.

Antigens, antibodies, therapeutic agents, targets, or complexes thereof may be driven through the channel by electroosmosis at 10, 25, 50, 75, 100, 125, 150, or 200 V/cm (including all values and ranges there between) excited with a laser, and detected with a confocal microscope or by confocal spectroscopy. Other means may also be used to drive the conjugates through the nanochannels, such as pressure based devices, centrifugal force, hydrostatic, and gravity based drivers. Microfluidic devices for use with the methods described herein will comprise a microfluidic chip or wafer holder, a voltage control model, an autofocus module, and an imaging system such as a confocal microscope or confocal spectroscopy system such as the Alba FCS spectrometer (ISS Inc.).

2. Detectable Moiety

In certain aspects, a detectable moiety is conjugated to a therapeutic agent, target, antibody, antibody fragment, or antigen for its subsequent detection either alone, or as a member of a complex. A detectable moiety will either directly or indirectly emitting a detectable signal.

Label selection plays an important role in single molecule studies. Ionic dyes, the standard choice in fluorescence microscopy assays, have several advantageous properties that make them well suited to single molecule studies. These include fluorescence quantum efficiencies approaching unity and fluorescence lifetimes below 10 ns. Accordingly, xanthene dyes such as Rhodamine 6G and tetremethyl-rhodamine isothyiocyanate are commonly used in single molecule studies. It is also becoming increasingly popular to use naturally occurring fluorescent proteins in single molecule studies. For example, Green Fluorescent Protein is resistant to photobleaching because its chromophore is located within the interior of its “β-can” structure and is thus protected from molecular oxygen. An alternative to organic dyes is semiconductor nanoparticles or quantum dots.

Label selection is guided primarily by the necessity to have a signal to noise ratio large enough that single molecules can be detected and analyzed. In multicolor experiments there are considerations beyond the basic ability to detect single molecules, such as the spectral properties of the fluorescent labels. In order to isolate fluorescence emission from a single species of fluorophore, the Stokes shift must be large enough to resolve the emission and excitation peaks. In a multicolor experiment, this situation can be complicated by multiple fluorescent species with overlapping emission and excitation spectra. Typically, when standard organic fluorophores are used, overlap in the emission spectra is managed by restricting the spectral range of collected fluorescence. However, this may result in rejected signal and reduced detection efficiency.

Suitable labels or dyes or fluorophores include, without limitation, quantum dots, PE, PE-Cy5, PE-Cy7, APC, APC-Cy7, Qdot 565, qdot 605, Qdot 655, Qdot 705, green fluorescent protein (GFP), eGFP, TurboGFP, TagGFP2, mUKGEmerald GFP, Superfolder GFP, Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSS-mOrange, CyOFP1, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP657, TagRFP675, mKate2, HcRed-Tandem, mPlum, mNeptune, NirFP, Kindling, far red fluorescent protein, yellow fluorescent protein (YFP), eYFP, TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKO_{K}, mKO2, mGrapel, mGrape2, zsYellow, eqFP611, Sirius, Sandercyanin, shBFP-N158S/L173I, near infrared proteins, iFP1.4, iRFP713, iRFP670, iRFP682, iRFP702, iRFP720, iFP2.0, mIFP, TDsmURFP, miRFP670, Brilliant Violet (BV) 421, BV 605, BV 510, BV 711, BV786, PerCP, PerCP/Cy5.5, Alexa Fluor dyes such as Alexa Fluor 350, 405, 430, 488, 514, 532, 546, 555, 568, 594, 633, 635, 647, 660, 680, 700, 750, and 790, FITC, BV570, BV650, DyLight 488, Dylight 649, and PE/Dazzle 594.

Compared to standard organic fluorophores, quantum dots have narrow and symmetrical emission spectra. Quantum dots also display a large effective Stokes shift, and different quantum dots can be excited by the same excitation source, typically in the blue part of the spectrum. The union of these traits results in the ability to simultaneously excite several species of quantum dots, or combinations of quantum dots and organic fluorophores, with a single light source, while the emission spectra are easily and entirely resolved. This increased detection efficiency is particularly relevant in single molecule detections where signal to noise ratio is often a limiting factor. Quantum dots are nanometer scale particles that absorb light, then quickly re-emit the light but in a different wavelength and thus color. The dots have optical properties that can be readily customized by changing the size or composition of the dots. Quantum dots are available in multiple colors and brightness, offered by either fluorescent dyes or semiconductor LEDs (light emitting diodes). In addition, quantum dot particles have many unique optical properties such as the ability to tune the absorption and emission wavelength by changing the size of the dot. Thus different-sized quantum dots emit light of different wavelengths. Quantum dots have been described in U.S. Pat. No. 6,207,392, and are commercially available from Quantum Dot Corporation. Quantum dots are defined in more detail in U.S. Patent Publication 20070166743, which is incorporated herein by reference in its entirety.

Fluorescence correlation spectroscopy (FCS) is based upon the recognition that as a fluorescently labeled molecule passes through a confocal laser beam and is excited, it emits photons of fluorescent light. The length of each photon burst is dependent upon the time spent in the confocal beam, and is diffusion controlled. By measuring the time associated with each burst, diffusion coefficients can be calculated, allowing discrimination of fluorescent molecules, such as bound and free species in a solution. Quantitation of free and bound ligand therefore allows determination of absolute concentrations of fluorophores and degree of binding. FCS is insensitive to miniaturization and therefore useful for implementation in microfluidic devices. When utilized with the present devices, a confocal laser is oriented such that the beam it emits is directed towards the detection section. The fluorescent detector is positioned to receive the photons of emitted light received from the detection section.

Single Molecule and Single Cell Measurements: Certain detection units that can be utilized with the systems described herein permit the detection and measurement of single molecules or cells. This capability can enable one to study processes that might not be apparent when making measurements of ensemble averages of populations of molecules or cells. In particular, such measurements allow observation of subpopulations of events within apparently homogeneous systems, and the analysis of dynamic events occurring on different time scales that would be lost upon averaging (see, e.g., Ishii, Y. and Yanagida, T. (2000) Single Mol. 1: 5-16 and Weiss S. (1999) Science 283: 676-1683. Fluorescence Correlation Spectroscopy (FCS; described supra) is one example of an intrinsically single molecule detection technique in which such detection units are useful. However, with standard optics, one can readily detect events at the single molecule or single cell level in essentially all of the modes described above (fluorescence intensity, fluorescence polarization, fluorescence resonance energy transfer (FRET), and fluorescence correlation spectroscopy (FCS)).

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials and Methods

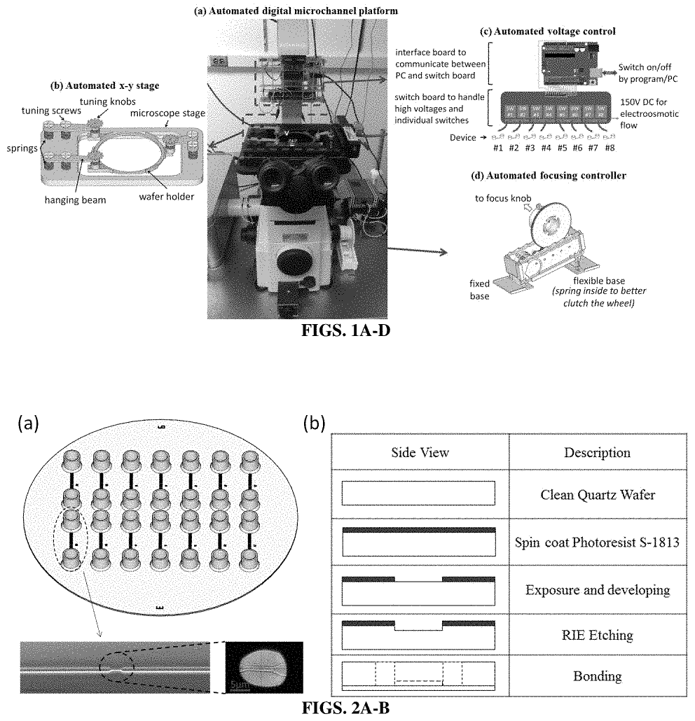

The working strategy for the flow-proteometric platform for analyzing protein concentration (FAP) is summarized in four steps (FIG. 1). First, a fixed cell sample or an FFPE tissue sample is treated with the designated fluorescence-conjugated primary monoclonal antibody to label the target proteins followed by the lysis process. Second, the sample solution is dispensed onto a microchannel on a silica wafer, and the electroosmosis force (EOF) then pumps the solution into the microchannel at a steady velocity. Third, autofocusing is applied to optimize the fluorescence detection. The final step is to record the fluorescence burst signals that represent single protein molecules passing through the detection volume. The numbers of fluorescence bursts are counted and later converted into a protein concentration. The details are described below.

Components of the Automated Digital Counting Platform—

The integrated digital counting platform is comprised of an adjustable wafer holder, a voltage control module, an autofocus module, and a platform that is integrated into an FCS spectrometer (Alba, ISS Inc.) (FIG. 1A). Each hanging beam is fixed in place by knobs and screws. Springs are equipped underneath the hanging beams to adjust and maintain the level of the wafer holder to horizontal (FIG. 1B). The voltage control module (FIG. 1C) is used to control the EOF and the flow rate in the microchannel. The voltage control module is comprised of two sections: a relay switchboard and an interface board. The relay switchboard triggers EOF in each microchannel and enables the proteins in the cell lysate to flow through the microchannel. The interface board communicates with the computer programs and the relay switchboard. An autofocus module (FIG. 1D) regulates the focus along the z-direction, which is integrated with gears and motors and controlled by the computer program. Motors control the focus knob through gears to optimize the microchannel z position for detection.

Microchannel Device—

The microchannels were made on an ultraviolet grade fused silica wafer using photolithography and wafer bonding as described previously (Chou et al., 2016). In brief, a single-molecule-detection microchannel was constructed on a 500-μm-thick quartz wafer through photolithography; this type of wafer was chosen for its minimum autofluorescence in the detection channel. FIG. 2 shows the fabrication flow chart for microchannels. First, the quartz wafer was cleaned with piranha solution for 5 minutes, followed by being spin-coated with a 1.5 am thin film of photoresist (Photoresist MICROPOSIT S1813). Then, microchannels were defined by photolithography on the photoresist. This pattern consisted of two holes for paired inlet and outlet reservoirs in each microchannel. Subsequently, the exposed area of the wafer was treated with CHF3 plasma to make 500-nm deep trenches connecting the reservoirs by reacted-ion etching. Finally, this quartz wafer containing micro trenches and holes for inlet and outlet reservoirs was temporarily bonded with a 170-am-thick plain quartz wafer that were cleaned in advance with piranha solution. After complete evaporation of solution, the temporarily bound wafers were placed into a furnace for 10 h at 1050° C. to permanently fuse wafers together and form a closed microchannel.

Operation of the Flow-Proteometric Platform for Analyzing Protein Concentration (FAP)—

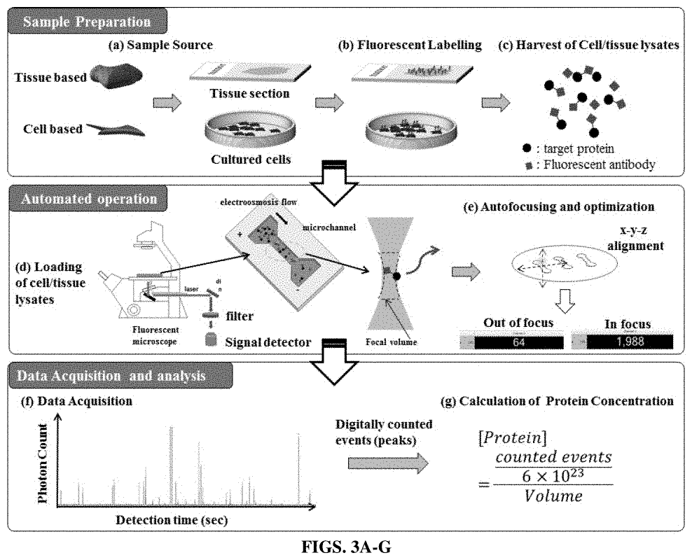

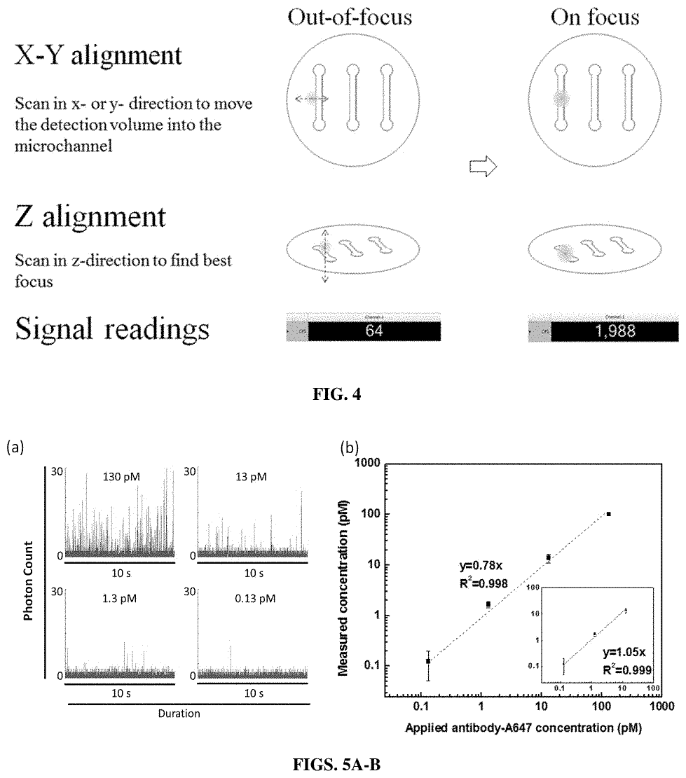

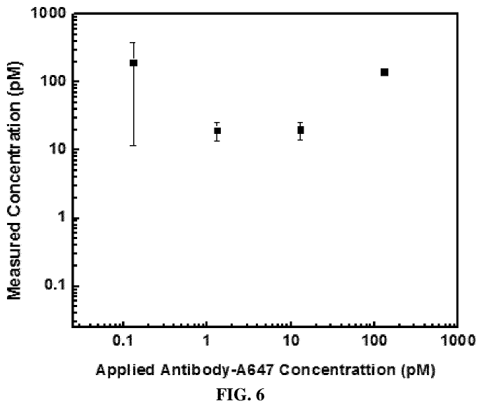

The FAP operating process consists of three major steps: sample preparation (FIGS. 3A-3C), sample flow in the microchannel (FIGS. 3D-3E), and data recording (FIGS. 3F-3G). The sample is prepared from cell or tissue lysates as described previously (Chou et al., 2014), and then loaded onto the microchannel. Subsequently, 150V potential is applied from both inlet and outlet reservoirs for triggering EOF inside the microchannel to transport proteins through the detection spot. In the beginning phase, the relay switch triggers the designated microchannel (microchannel #1) for the first experiment. Meanwhile, a UV laser with 405-nm wavelength also excites the autofluorescence of the sample solution, which enables the system to identify the position of the microchannel. The focusing process consists of two steps, x-y plane and z-direction adjustments. In the first step, the microchannel is moved to the approximate detection position, and the program executes the optimization process by finding the x-y position with the highest fluorescence photon count. Next, the focusing process optimizes the position along the z-direction based on the signal intensity. FIG. 4 illustrates the process of auto-optimization in detail. Once the microchannel position is optimized based on the maximum fluorescent signal intensity, the UV 405-nm laser is removed and then replaced by a UV 635-nm laser to excite the fluorescent label on the target molecule. Each target “event” (photon burst) is identified and counted when it flows through the detection volume. For each detection, 2 min is sufficient to collect the events but it can be extended as needed. Once data acquisition is completed, the control program announces the movement of the stage to the next microchannel and turns on the relay switch for initiation of the next sample analysis.

Cultured Cells and Human Breast Tumor Tissue Samples—

HeLa and MDA-MB-231 cells were obtained from the American Type Culture Collection and cultured in Dulbecco's modified Eagle's medium/F-12 medium with 10% fetal bovine serum and 100 ag/ml penicillin and streptomycin; cells were negative for mycoplasma contamination. For exogenous PD-L1 expression, HeLa cells were transfected with a pCDH-lentiviral expression virus, which contained human PD-L1 cDNA (shRNA and ORFeome Core, MD Anderson Cancer Center). Subclones with stable PD-L1 expression were selected using puromycin (InvivoGen, San Diego, Calif., USA). The expression of PD-L1 in both HeLa and MDA-MB-231 cells was confirmed by Western blotting. Human breast tumor tissue specimens were obtained in compliance with relevant laws and institutional guidelines following the approval by the Institutional Review Board at UT MD Anderson Cancer Center. Specimens from two patients with breast cancer were tested. Written informed consent was obtained from each patient at the time of enrollment.

IHC Staining of Tissue Samples—

IHC staining was performed as described previously (Lee et al., 2007). Antigen retrieval was carried out by heating in 0.01 M sodium-citrate buffer (pH 6.0) using a microwave oven. To block endogenous peroxidase activity, the sections were treated with 1% hydrogen peroxide in methanol for 30 min. After 1 h preincubation in 10% normal serum to prevent nonspecific staining, the sections were incubated with the PD-L1 antibody (PD-L1 IHC 28-8 pharmDx, Agilent) at 4° C. overnight. The sections were washed with PBS and then incubated with an avidin-biotin-peroxidase complex. Visualization was performed using amino-ethylcarbazole chromogen. The intensity of staining was ranked according to histologic scoring.

Fluorescence Labeling and Sample Preparation—

Cultured cells were fixed in 1% paraformaldehyde at room temperature (RT) for 10 min, washed with phosphate-buffered saline (PBS), and treated with 2.5% Triton X-100 for 5 min at RT. Samples were then blocked by using 6% bovine serum albumin (BSA) in PBS for 1 hour at RT. After blocking, Alexa Fluor 647-conjugated PD-L1 antibody (28-8, Abcam) or isotype control IgG (Invitrogen) for designated experiments was diluted in BSA and incubated with samples overnight at 4° C. Samples were then washed with PBS to remove the unbound antibodies. Next, the samples were lysed and proteins extracted in radioimmunoprecipitation assay buffer (50 mM Tris-HCl [pH 7.4], 150 mM NaCl, 1% NP40, 1 mM EDTA) (Hus et al., 2011). Sample lysates were sonicated using a Bioruptor (Diagenode) at high intensity 10 times in 15-sec cycles to degrade the cell structure and diluted in detection buffer (20 mM Hepes-KOH [pH 7.9], 0.1 mM KCl, 2 mM MgCl₂, 15 mM NaCl, 0.2 mM ethylenediaminetetraacetic acid, 10% glycerol). Diluted samples were then loaded into the microchannel for detection of the amount of Alexa Fluor 647-conjugated antibody. For the FFPE sample preparation, fluorescence labeling procedures were similar to those for IHC staining, with antigen retrieval and 5% BSA blocking. The primary antibody was replaced by Alexa Fluor 647-conjugated antibody. After the fluorescent-antibody-labeling process, the tissue samples were lysed using radioimmunoprecipitation assay buffer and sonicated at high intensity 10 times in 30-sec cycles to break the tissue structure. Samples were then diluted in the detection buffer as described above.

Determination and Calculation of Protein Concentration—

Estimation of the concentration of fluorescence-labeled proteins in FAP was the combination of digital events counting and FCS-based sample volume fitting determination. In brief, photon burst raw data were collected at a rate of 100 kHz, and each data point represents the photo count within 10 ms. The data was further compressed to 1 kHz for burst calculation. First, this noise level was defined by dividing the compressed data file into several 100-point long sections sequentially. Second, for each section, the average (m), SD (σ), and local noise level (m+3σ) were calculated. Third, another event selection line that was three times higher than the basal noise level was applied to filter out lower and ambiguous bursts. Only the bursts higher than the event threshold line were viewed as valid burst signal events and counted. For the FCS-based sample volume fitting, the autocorrelation function of the actual data series is based on the equation (1) (Kohler et al., 2000).

$$G ( τ ) = ( \frac{1}{π \sqrt{π} {w}_{0}^{2} {z}_{0} 〈 C 〉} ) \frac{1}{( 1 + \frac{r D τ}{{w}_{0}^{2}} ) \sqrt{1 + \frac{4 D τ}{{z}_{0}^{2}}}} exp [ \frac{- {( V τ )}^{3}}{{w}_{0}^{2} {z}_{0} ( 1 + \frac{4 D τ}{{w}_{0}^{2}} ) \sqrt{1 + \frac{4 D τ}{{z}_{0}^{2}}}} ] \qquad ( 1 )$$

w₀: beam width (μm), z₀: microchannel height (μm), C: concentration of fluorescence species (nM), D: diffusion coefficient of fluorescence species (μm2/sec), and V: flow velocity (m/sec).

The flow velocity, which was required to estimate the detected sample volume, can be obtained by fitting the equation (1) with known parameters. To minimize the variation in quantification, the flow velocity is derived from FCS fitting for each 10-s recording window. The two highest and two lowest flow velocities were ignored in order to minimize the variation. The averaged value is used as the global flow velocity of the sample for calculation of the protein concentration. Subsequently, using the global velocity and the dimension of the microchannel, the volume flow rate can be calculated. Together with the number of fluorescence bursts counted in a given time, the protein concentration can be approximated using the equation (2).

$$Concentration ( pM ) = \frac{( \frac{burst numbers}{6.02 \times {10}^{23}} )}{w \times h \times v \times t} \times {10}^{27} \qquad ( 2 )$$

where w: microchannel width (am), h: microchannel height (μm), v: flow velocity (m/sec), and t: detection time (sec).

DQON Cell Culture and Xenograft Model—

CHO cells obtained from the American Type Culture Collection were cultured in Dulbecco's modified Eagle's medium/F-12 medium with 10% fetal bovine serum and 100 ag/ml penicillin and streptomycin. EGFR^{GFP} was transfected into CHO cells, and the stable EGFR^{GFP}-expressing clone (CHO-EGFR^{GFP}) was selected by G418 (500 μg/ml). Full-length EGFR-GFP cDNA expression was confirmed by Western blot analysis. For EGF stimulation, CHO-EGFR^{GFP} cells were incubated with EGF (50 ng/ml; Sigma) for 30 minutes before the anti-EGFR antibody treatment. GEO-EGFR^{GFP} cells were generated as described previously (Liao et al., 2015). Nude mice were injected with 5×10⁶ GEO-EGFR^{GFP} subcutaneously. After the tumor reached 8 mm in diameter, EGFRab^{A647} (100 μg) or IgG (one mouse each) was administrated intravenously, and small pieces of tumor tissue were collected at the indicated times. All animal experiments were carried out in accordance with a protocol approved by MD Anderson's Institutional Animal Care and Use Committee (068706139), and the study adhered to NIH guidelines for the use of research animals.

DQON Sample Lysate Preparation—

EGFRab^{A647} was prepared by using a fluorescence antibody labeling kit (ThermoFisher) to directly conjugate A647 to cetuximab. For the cell culture sample, 1×10⁵ cells were treated with EGFRab^{A647} and washed with PBS. The cells were then lysed with 10 μl of radio-immunoprecipitation assay buffer (50 mM Tris-HCl [pH7.4], 150 mM NaCl, 1% NP40, 1 mM ethylene diamine tetraacetic acid), mixed thoroughly, and incubated on ice for 20 minutes. The lysates were then centrifuged at 16,000×g for 1 minute to remove large debris, and the supernatant was collected. About 1 μg of lysate was diluted in 500 μl of detection buffer (20 mM HEPES-KOH [pH7.9], 0.1 mM KCl, 2 mM MgCl₂, 15 mM NaCl, 0.2 mM ethylene diamine tetra acetic acid, 10% glycerol) for analysis. Tissue samples were degraded with homogenizer with lysis buffer, incubated on ice for 20 minutes, and then prepared for analysis as described for cell cultures.

DQON Optical System—

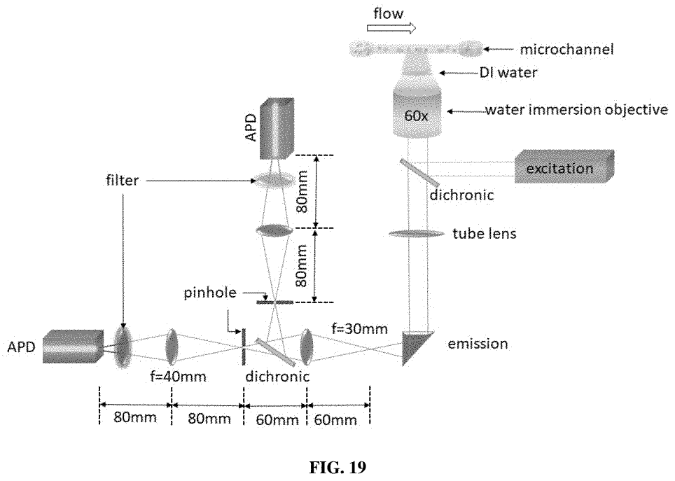

The schematic diagram of DQON is shown in FIG. 19. The sample lysate was loaded into each of the microchannel's reservoirs, and 150 V potential was applied to generate electroosmotic flow. The electrical conductivity of the sample solutions was maintained to ensure that the flow velocity was constant for all detections (Kameoka et al., 2001). Before each analysis, the sample lysate was first allowed to flow through the microchannel for 20 minutes to ensure that the flow was sufficiently stable for single molecule detection. Two excitation lasers with wavelengths of 488 and 635 nm were focused through a 60× water immersion objective (NA 1.2) at the center of the microchannel. As each protein complex passed through the focus spot, time-coincident fluorescence emitted from the components of the complex were detected using a single-molecule spectroscope (Alba, ISS) equipped with 2 sensitive avalanche photodiodes for observing green (GFP) and red (A647) photon signals. The system included specific optical filters (531/40 and 685/40) to prevent signal bleed-through between different fluorophores during detection.

Determination of Target Protein and Antibody Drug Events—

The determination of effective events was processed as described previously (Chou et al., 2016). In brief, raw data were collected at a rate of 100 kHz, with each data point representing the photon count within 10 tsec. Before a valid photon burst signal can be detected, a basal noise level has to be defined so that the start and end time of a burst can be located; therefore, the basal noise level was defined by first dividing the raw data file into several 100-point-long sections sequentially. For each section, the average (m), standard deviation (σ), and local noise level (m+3G) were calculated. The minimum 10% of these local noise levels were selected. The average of the selected noise levels then served as the basal noise level. Another event-selection line that was 3 times (for cell samples) or 5 times (for tissue samples) higher than the basal noise level was applied to filter out auto-fluorescence and ambiguous bursts. Only the bursts higher than the event-selection line were considered to be valid burst signal events and counted. The term “event” is used to specify such a burst when it is associated with a molecule. A coincidence of 2 color bursts (events) was defined as any overlapping bursts (events) of 2 different colors. The numbers and ratios of 2-color coincidences were calculated based on the above definitions.

On-Target Interaction Quantification—

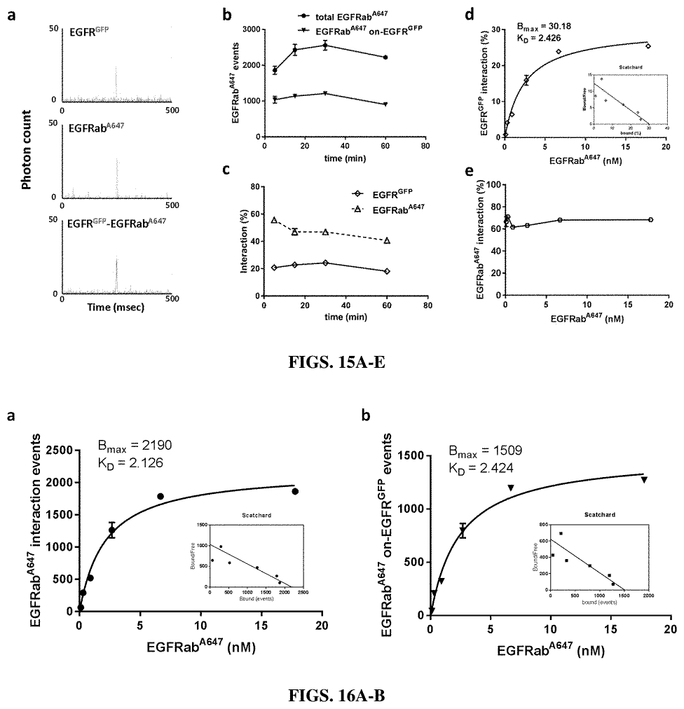

Individual target proteins, antibodies, and antibody-protein complexes corresponded to different photon bursts, which were defined as events. Representative lone EGFR^{GFP} event, lone EGFRab^{A647} event, and EGFR^{GFP}-EGFRab^{A647}“on-target” event are shown in FIG. 15A. Each analysis collected 5,000 EGFR^{GFP} events. EGFRab^{A647} and EGFR^{GFP}-EGFRab^{A647} events occurring within the period that 5,000 EGFR^{GFP} events were collected were counted. The interaction ratios were determined from either the EGFR^{GFP} or EGFRab^{A647} events that involved interaction as follows:

$${EFFR}^{GFP} interaction ratio = \frac{{}^{″} on {target}^{″} events}{total {EGFR}^{GFP} events} \times 100 \%$$

$${EGFRab}^{A 647} interaction ratio = \frac{{}^{″} on {target}^{″} events}{total {EGFRab}^{A 647} events} \times 100 \%$$

where “total EGFR^{GFP} events” are EGFR^{GFP}-EGFRab^{A647} events plus lone EGFR^{GFP} events (around 5,000 events) and “total EGFRab^{A647} events” are EGFR^{GFP}-EGFRab^{A647} events plus lone EGFRab^{A647} events in cells. In addition, “total EGFRab^{A647} events” is the EGFRab^{A647} events that left in cells after treatment.

Determination of the Dissociation Constant—

The disassociation constant (K_{D}) was determined with a interaction curve assay and the formula

$$B = \frac{{B}_{max} \times F}{{K}_{D} + F}$$

where B is the concentration of the antibody-target complex, Bₘₐₓ is the total number of interaction sites for a target in cells or tissue, F is the concentration of free ligand, and K_{D} is the disassociation constant.

In this study, the ligand against EGFR^{GFP} was cetuximab conjugated with A647 (EGFRab^{A647}). Different concentrations of EGFRab^{A647} (0.1-17.77 nM) were used to treat CHO-EGFR^{GFP} cells. After 5 minutes of treatment, cell lysates were collected and loaded into the microchannel for DQON analysis, and the K_{D} was acquired.

### Example 2—Standard-Free Determination of Protein Concentration

Advanced targeted therapy, including immune checkpoint therapy, has greatly improved cancer treatment strategies. These therapies aim at specific target molecules to suppress tumor growth by blocking the relevant signaling pathways. However, it is critical to apply the therapy to cancer cells with the target expression because treatment in the absence of the target may cause a worse prognosis (Spigel et al. 2013). Therefore, many targeted therapies require evaluating the targets in cancer cells before administering the treatment (Buter and Giaccone 2005; Engel and Kaklamani 2007), and methods for assessing the amount of target protein in cancer tissue can support clinicians in stratifying patients for the best treatment strategies. Although IHC is the major approach to determine the target protein levels, obtaining the protein concentration information can further support pathologists and clinicians in accurately diagnosing the disease and determining the treatment plans.

To verify whether the FAP platform can accurately measure protein amounts and concentrations, diluted Alexa Fluor 647-conjugated IgG samples were prepared (IgG concentration ranging from 0.13 to 130 pM) and tested on FAP. As Alexa 647 provided an excellent signal-to-noise ratio (˜10) in the microchannel-based detection, individual photon burst signal, which was higher than the threshold level, could be clearly identified and counted (FIG. 5A). The measured concentration was highly correlated with the actual concentration in the control samples, showing a coefficient of determination (R²) of 0.998 (FIG. 5B). Although the correlation equation is y=0.78x, it is improved to y=1.05× when the range is narrowed from 0.13 to 13 pM. The possible reason for the difference is that the saturated photon bursts from highly concentrated samples result in an underestimation of burst numbers. Meanwhile, FAP was compared with FCS by testing the same samples under the same conditions. A higher concentration of Alexa Fluor 647-IgG, such as 130 pM, was precisely predicted by both conventional FCS and FAP. However, the FAP platform successfully measured the low-concentration samples (<13 pM), whereas FCS failed to provide accurate results (FIG. 6). This is because FCS is particularly noisy when measuring the low-concentration samples (Enderlein et al., 2004; Levin and Carson, 2004; Slaughter and Li, 2004). Thus, the FAP flow-proteometric platform disclosed herein provided a standard-free protein concentration measurement, and was particularly effective for low-concentration samples (<13 pM). This result indicates that the automated flow-proteometric platform could accurately reflect the real sample concentration for absolute concentration measurement without correlation with a standard, which is much simpler and less time consuming than the conventional methods such as enzyme-linked immunosorbent assay (ELISA).

### Example 3—Determination of PD-L1 Concentration in Cancer Cells

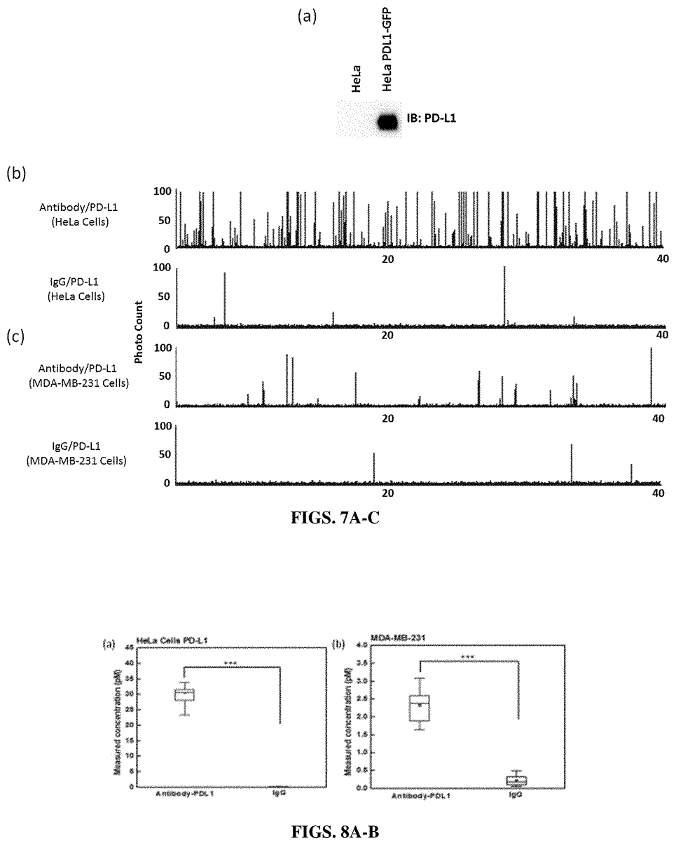

As mentioned previously, the amount of PD-L1 in cancer cells serves as an important biomarker to predict the response to the immune checkpoint therapy (Yuasa et al., 2017). However, standard PD-L1 measurement by IHC is complicated. A method for direct protein concentration measurement in tissue could improve the evaluation of PD-L1 levels. Therefore, the FAP platform's utility for measuring concentration of a known protein in a cell was tested next. First, PD-L1 was overexpressed in HeLa cells (FIG. 7A), and labeled with Alexa Fluor 647-conjugated PD-L1 antibody. After applying the wash buffer to remove the unbound PD-L1 antibody, the cells were then lysed and sonicated to break down the cell structure for single PD-L1 molecule counting. FIG. 7B shows the individual fluorescence events for PD-L1 and negative control (isotype IgG-Alexa647) in a 40-sec detection period. The PD-L1 fluorescence burst counts were significantly increased compared to those of the control IgG, indicating the great majority of PD-L1 events were indeed PD-L1 proteins in cells. The concentration of PD-L1 in the lysate was calculated to be around 30.5 pM (FIG. 8A).

In addition to the exogenous PD-L1 detection, endogenous PD-L1 was measured in cancer cells. It has been previously been reported that breast cancer cell line MDA-MB-231 highly expresses PD-L1 on the cell surface membrane (Chemnitz et al., 2004; Yuasa et al., 2017), thus it was determined to be an ideal sample to measure the endogenous PD-L1 concentration using the FAP platform. Similar to the analysis of exogenous PD-Li expressed in HeLa PD-L1 cells, endogenous PD-L1 in MDA-MB-231 cells was first labeled with the Alexa Fluor 647-conjugated PD-L1 antibody, and then cells were washed to remove the unbound antibody before lysis. PD-L1 labeled MDA-MB-231 cell lysate was loaded onto the FAP platform, and each individual PD-L1 antibody photon burst was recognized and counted (FIG. 7C). After converting the events number into the molar concentration, the PD-L1 concentration in MDA-MB-231 cell lysate sample was determined to be 2.33 pM, whereas non-specific isotype IgG was recognized as 0.22 pM (FIG. 8B). This result clearly demonstrates the specificity and sensitivity of the FAP platform.

Since the measured protein concentrations reflected the lysate concentration in lysis buffer, the protein concentration was further converted to the original concentration in cells. To determine the PD-L1 concentration in HeLa PD-L1 cells the following values were used: cell volume per cell: 4.7 pL; cell number per sample: 5×10⁴; total volume of cells: 0.235 μL. The measured concentration of PD-L1 using the FAP platform: 30.5 pM. Since the lysate was diluted nine times before analysis, the concentration of PD-L1 before using microchannel was calculated to be: 30.5 pM×9=274.5 pM. Therefore the concentration of PD-L1 from tissue lysate was determined as follows:

$$\frac{{[ PD - L 1 ]}_{HeLa} \times 0.235 µL}{300 µL} = 274.5 pM$$

Thus the concentration of PD-L1 per cell is: [PD−L1]_{HeLa}=357.1 nM.

PD-Li concentration of from MDA-MB-231 cells was determined using the following values: Cell volume per cell: 5.7 pL; cell number: 5×10⁴; total cell volume: 0.285 μL. The concentration of PD-L1 was measured by FAP to be: 2.33 pM. Since the lysate was diluted five times prior to analysis, the concentration of PD-L1 before using microchannel was calculated to be: 2.33 pM×5=11.65 pM.

The total concentration of PD-L1 from tissue lysate was determined using the following equation:

$$\frac{{[ PD - L 1 ]}_{MDA - MB - 231} \times 0.285 µL}{300 µL} = 11.65 pM$$

Thus the concentration of PD-L1 per cell is: [PD−L1]_{MDA-MB-231}=12.3 nM. Determination that the PD-Li concentrations in single HeLa PD-L1 cells and MDA-MB-231 cells were 357.1 nM and 12.3 nM, respectively (FIG. 9) provide proof that absolute protein concentration in cells can be measured directly without the requirement of a standard.

### Example 4—Determination of PD-L1 Concentration in FFPE Tumor Tissue Specimens

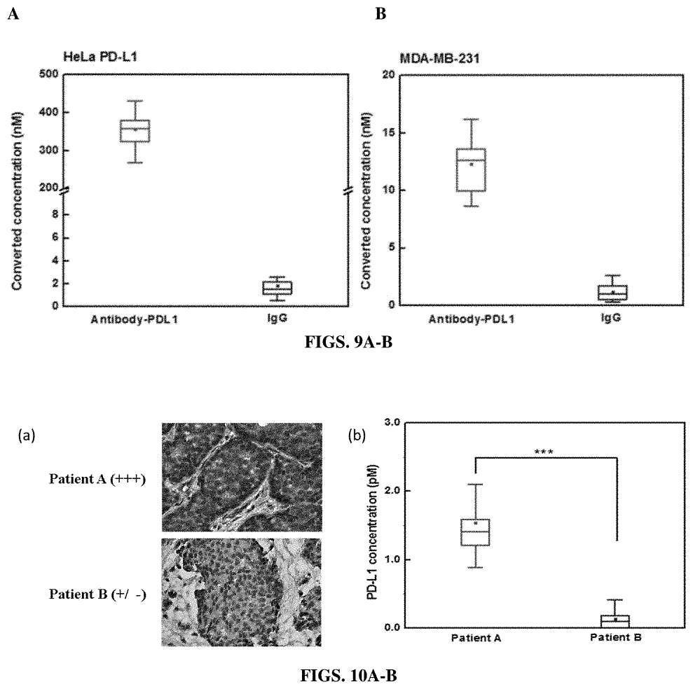

Currently, IHC staining is the standard procedure for analyzing the target protein amount in FFPE tissue for diagnostic purposes. However, this process is laborious and time consuming, and there are presently no efficient methods of measuring the target protein concentration directly from FFPE tissue available. To address this issue, the FAP platform was tested to determine whether it can be used to measure the target protein concentration directly in the absence of using purified target molecules as a standard. FFPE tumor tissue samples from two breast cancer patients were used to perform both conventional PD-L1 IHC staining and digital protein concentration analysis using the FAP platform. As shown in FIG. 10A, patient A showed (+++) signal intensity in IHC staining, whereas patient B showed (+/−). Slides containing tissue that was adjacent to the area used for IHC staining were dissected then labeled with the Alexa Fluor 647-conjugated PD-L1 antibody, and PD-L1 concentration was measured using the FAP platform. As expected, more photon bursts were observed in patient A's sample than in patient B's (FIG. 11A).

PD-L1 photon events from FFPE tissue sample A were converted into concentrations using the following information: The lysates from tissue samples containing high or low levels of PD-L1 were diluted 5 times from the original solution. Area of tissue: 77.888 mm²; thickness: 5 am; total volume of tissue: 0.38944 μL. The measured concentration of PD-L1 in the microchannel was measured to be: 1.39 pM (FIG. 10B), while the concentration of PD-L1 before using microchannel was found to be: 1.39 pM×5=6.95 pM. Thus the concentration of PD-L1 converted from analytic solution to tissue sample was determined as follows:

$$\frac{{[ PDL 1 ]}_{High PDL 1 tissue} \times 0.38944 µL}{300 µL} = 6.95 pM$$

Therefore, the concentration of PD-L1 in the tissue from patient A was [PDL1]_{High PDL tissue}=5.35 nM (FIG. 11B).

PD-L1 photon events from FFPE tissue sample B were converted to concentrations using the following information: Area of tissue: 44.926 mm²; thickness: 5 μm; total volume of tissue: 0.22436 pL. The measured concentration of PD-L1 using microchannel: 0.11 pM (FIG. 10B). Concentration of PD-L1 before using microchannel: 0.11 pM×5=0.55 pM. The concentration of PD-L1 converted from analytic solution to tissue sample was calculated as follows:

$$\frac{{[ PDL 1 ]}_{Low PDL 1 tissue} \times 0.22436 µL}{300 µL} = 0.55 pM$$

Therefore, the concentration of PD-L1 in the tissue from sample B was [PDL1]_{Low PDL1 tissue}=0.73 nM (FIG. 11B).

As shown above, the PD-L1 concentration from sample lysates into concentrations for the original tissue yielded 5.35 nM and 0.73 nM PD-L1 concentrations for patients A and B, respectively (FIG. 11B). These results demonstrated that protein concentration in FFPE tissue can be directly quantified and the results pattern are consistent with those of conventional IHC staining. Importantly, FAP provided the tissue PD-L1 concentration information which can support the pathologists to avoid the bias from tissue IHC staining and reading.

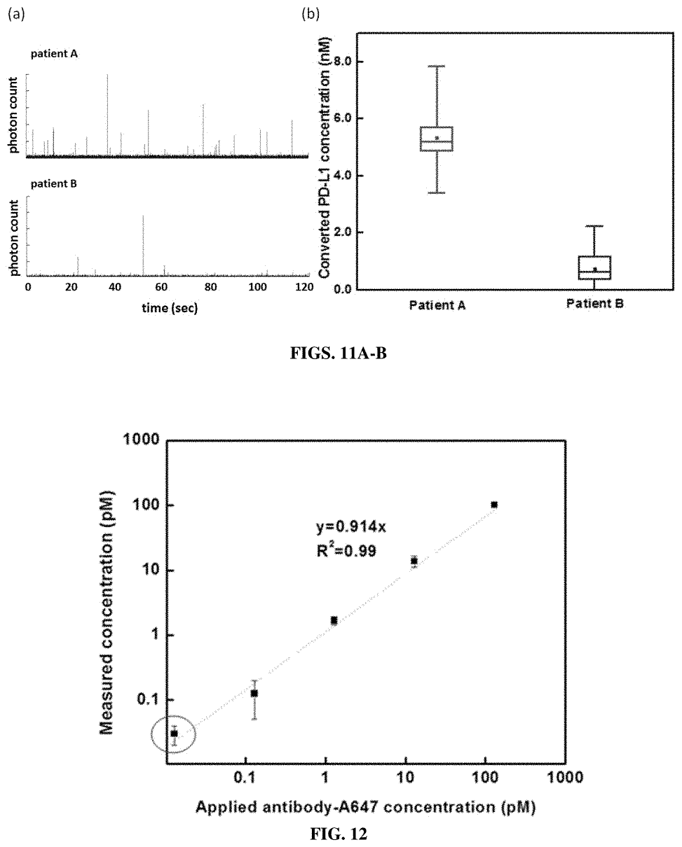

The concentration range demonstrated in these studies is from 0.13 to 130 pM for a 2-minute analysis. For protein concentrations <0.13 pM, they can be measured by extending the detection time and increasing the flow speed. For instance, protein concentration with 13 fM was measured with the increment of EOF voltage to 400V and the extension of measuring time to 8 minutes (FIG. 12).

Protein concentration critically affects the cellular function. Although conventional methods such as Western blotting can demonstrate a semi-quantitative difference between endogenous and exogenous protein expression, the data provided herein show quantitative evidence that PD-L1 protein concentration in an overexpression system can be 30 times higher than the endogenous expression in cancer cells. Therefore, caution should be used when adopting an exogenous approach to investigate protein function. However, this protein concentration method can be applied to other proteins.

Since the concentration is determined by the amount of fluorescence-conjugated antibody associated with the target protein, one should be aware that steric hindrance and binding position of the antibody may reduce the accuracy of the detection, as with all immune-staining methods (Zhang et al., 2005; Peng et al., 2016). As this effect occurs in all immune-staining methods, such as immunofluorescence staining, IHC, and the FAP analysis presented herein, the results obtained by these methods are dependent on the antibody been used. Therefore, selecting the appropriate antibody that can label most of the target protein in its native form can improve the accuracy of the measurement. Anti-PD-L1 antibody clone 28-8, used in this methods provided herein, was approved by FDA for the determination of the PD-L1 level by IHC analysis, thus the PD-L1 concentrations measured herein are a reflection of this antibody's target labelling ability. Also, the non-specific binding of antibody to cell or tissue sample may limited the sensitivity of the detection. An appropriate antibody non-specific binding control is helpful to determine the basal noise level.

The results presented herein indicate that that the FAP platform can measure the PD-L1 concentration directly, whether from diluted proteins, cells, or even notoriously challenging FFPE tissue samples, and support the IHC staining results by providing the actual protein concentration. This information can be helpful for pathologists to better determine the protein level in tissue because it avoids the variation from the staining development process and the reading bias from different person. In addition, since the FAP is a fluorescence-based digital quantification platform, it is fully capable of performing the multiplex analyses. Multiple fluorescent conjugated antibodies can potentially be applied to label different target proteins in the tissue sample simultaneously. Therefore, it is possible to measure different targets in a single tissue in one setting. Also, by integrating a microdissection system, measurement of the target protein concentration in a specific tissue area is also feasible. All these demonstrate the potential clinical and basic research applications of FAP.

### Example 5—Digital Quantification of On-Target Binding Constant (DQON)

Targeted therapeutic agents, such as monoclonal antibodies, are widely used in the treatment of a variety of diseases. An ideal therapeutic agent has high sensitivity and specificity for its target, and will tightly associate with the target at a low dose with minimum association with non-targets. To determine the sensitivity and specificity of an antibody, researchers use various methods, such as enzyme-linked immunosorbent assay (Friguet et al., 1985), surface plasmon resonance (Hahnefeld et al., 2004), and biolayer interferometry (Shah et al., 2014), for purified sample interaction analysis and dissociation constant (K_{D}) measurement. Antibodies selected by these methods are then validated by cell-based methods, such as Western blotting (Signore et al., 2017), flow cytometry (Chosy et al., 2003), and Cas9 gene knock-out analysis (Zotova et al., 2016). However, most of these methods are qualitative or only semi-quantitative and cannot detect antibody-target interaction directly, which makes it difficult to quantify such interaction in cells. Therefore, a cell-based quantitative method for directly assessing antibody on-target interaction is critical to the development of effective therapeutic antibodies.

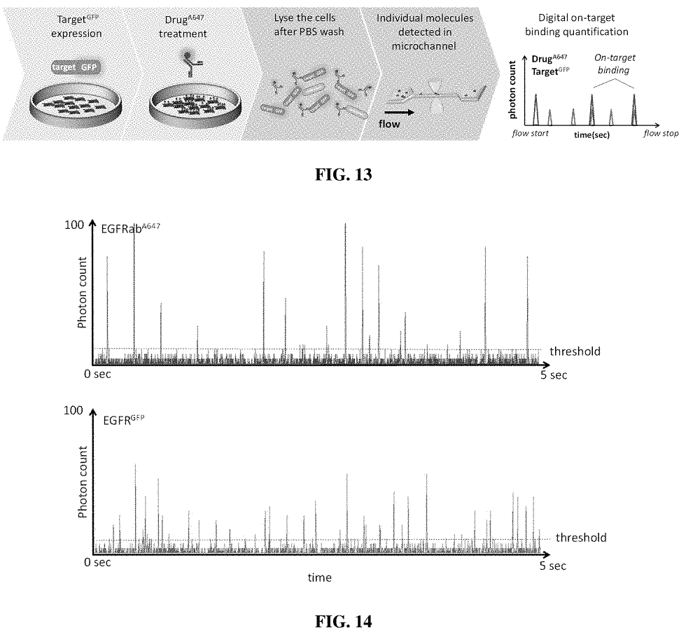

Observing antibody on-target interaction at the single-molecule level would provide direct evidence of such interaction as well as a way to assess the amount of antibody that binds to its target. Digital Quantification for “ON-target” interaction (DQON) was developed in order to measure the therapeutic antibody interaction ratio and K_{D} in cultured cells and in cells obtained from xenograft model tissue samples. DQON is a simple, straightforward methodology that requires only a few cells and can be completed within a couple of hours (FIG. 13). In brief, cells expressing the target membrane proteins fused with the green fluorescence proteins (GFPs) were treated with Alexa Fluor 647® (A647)-conjugated antibodies. After treatment, unbound antibodies were removed with a phosphate buffered saline (PBS) wash, and the cells were lysed. Subsequently, the lysates were collected and subjected to DQON analysis. As the lysate flows through the microchannel's detection spot, each antibody- or target protein-generated photon burst is captured; coincident photon bursts indicate antibody on-target interaction. On-target interaction ratios are then determined by calculating the amount of target protein or antibody involved in interaction.

In this proof-of-concept study, a clinical anti-epidermal growth factor receptor (EGFR) therapeutic antibody (cetuximab; EGFRab hereafter) was used (Harding and Burtness, 2005). EGFR deregulation often causes cancer progression, and EGFRab, which has high sensitivity and specificity for EGFR interaction, has been used to treat colorectal cancer (Hutchinson et al., 2015), non-small cell lung cancer (Govindan, 2004), and head and neck cancer (Kim et al., 2004). First, cells were generated that stably express EGFR^{GFP} (CHO-EGFR^{GFP} cells). Next, the CHO-EGFR^{GFP} cells were treated with A647-conjugated EGFRab (EGFRab^{A647}). Following treatment, the cells were lysed, and the cell lysate was collected for DQON analysis. As the lysate flowed through the microchannel, individual photon bursts were observed from EGFRab^{A647} and EGFR^{GFP} (FIG. 14). A single green photon burst indicated EGFR^{GFP} with no EGFRab^{A647} interaction (FIG. 15A, top), whereas a red photon burst indicated EGFRab^{A647} with no EGFR^{GFP} interaction (FIG. 15A, middle). Coincident green and red photon bursts indicated EGFRab^{A647}-EGFR^{GFP} interaction (FIG. 15A, bottom).

EGFRab^{A647}-EGFR^{GFP} interaction was then assessed following different durations of treatment with EGFRab^{A647}. CHO-EGFR^{GFP} cells were treated with 20 nM EGFRab^{A647} for 5-60 minutes, washed with PBS to remove unbound EGFRab^{A647}, lysed, and analyzed with DQON. An average of 1,861 overall EGFRab^{A647} events were detected per 5,000 EGFR^{GFP} events after 5 minutes of treatment (FIG. 15b). Of these EGFRab^{A647} events, an average of 1,038 were EGFRab^{A647}-EGFR^{GFP} events. Extending the treatment time increased the number of overall EGFRab^{A647} events, which peaked 30 minutes after treatment. By interaction ratio, the proportion of EGFRab^{A647} that was bound to EGFR^{GFP} was nearly 60% at 5 minutes but only about 40% at 60 minutes (FIG. 15C). Interestingly, the ratio of EGFR^{GFP} involved in EGFRab^{A647} interaction did not differ during treatment. These results reflect the dynamics of EGFRab^{A647} in targeting EGFR^{GFP}.

Because the highest EGFRab^{A647}-EGFR^{GFP} interaction ratio was detected within the first 5 minutes of treatment, this time point was selected for assessing the extent to which EGFRab^{A647} concentration is correlated with on-target (EGFR) interaction. With different treatment concentrations, the interaction saturation curve was first plotted based on overall EGFRab^{A647} events (which indicated the association between EGFRab^{A647} and cells), and the K_{D} was found to be 2.126 nM (FIG. 16A). Next, the curve was plotted based on the EGFRab^{A647}-EGFR^{GFP} events, and the K_{D} was found to be slightly increased, to 2.424 nM (FIG. 16B). That the overall EGFRab^{A647} K_{D} was similar to the EGFRab^{A647}-EGFR^{GFP} K_{D} suggested that EGFRab^{A647} has less off-target interaction. Otherwise, the overall K_{D} might be affected by the off-target interaction.

The ratios of EGFRab^{A647} and EGFR^{GFP} involved in EGFRab^{A647}-EGFR^{GFP} interaction were then calculated. From the aspect of overall EGFR^{GFP}, the EGFRab^{A647}-EGFR^{GFP} interaction ratio K_{D} was 2.436 nM (FIG. 15D), which was identical to the K_{D} calculated by the number of interaction events (FIG. 16B). The maximum interaction (Bₘax) ratio was 30.18%, which suggested that high-dose EGFRab^{A647} targeted about one-third of the EGFR^{GFP} overexpressed in CHO cells. Interestingly, the interaction ratios of EGFRab^{A647} left in cells were all around 70%, regardless of EGFRab^{A647} original treatment concentration (FIG. 15E), which suggests that 70% of EGFRab^{A647} remained bound to EGFR^{GFP} in the cell lysate and the other 30% was either dissociated from EGFR^{GFP} or bound to a different protein. However, most of the 30% of non-EGFR-interaction events were not considered to be off-target, because the EGFRab^{A647} has little off-target interaction as described previously. Because one would expect that the ratio of an antibody with good blockade ability bound to the target would be high, these quantitative results demonstrate the efficacy of EGFRab^{A647}. These results, all of which were generated from one set of multi-concentration experiments, further demonstrate that DQON is a simple method for digitally quantifying antibody on-target interaction in living cells.

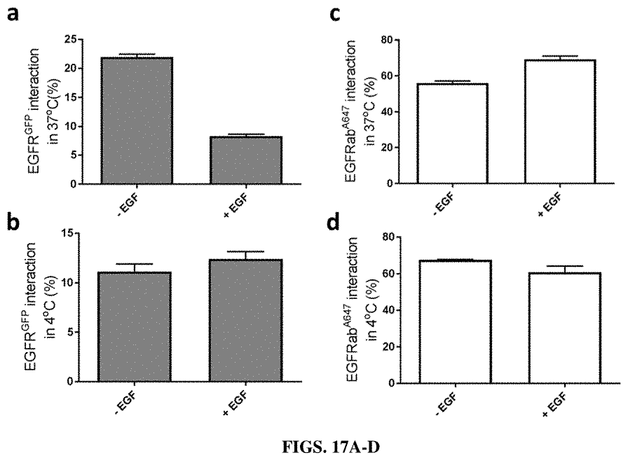

Next, the extent to which the EGFRab^{A647}-EGFR^{GFP} interaction ratio is affected by treatment with EGFR's ligand, EGF, was determined. CHO-EGFR^{GFP} cells were pretreated with or without 50 ng/ml EGF for 30 minutes and then treated with 15 nM EGFRab^{A647} for 5 minutes. After EGF treatment, the ratio of EGFR^{GFP} involved in EGFRab^{A647} interaction was decreased significantly (FIG. 17A). It was suspected that EGF triggered EGFR endocytosis, as reported previously (Lee et al., 2015), which consequently reduced the amount of surface EGFR that could be targeted by EGFRab^{A647}. To test this hypothesis, EGF-induced EGFR endocytosis was suppressed by lowering the temperature at which the cells were treated with EGF and EGFRab^{A647} to 4° C. With endocytosis suppressed, EGF did not affect the on-target interaction ratio, indicating that endocytosis was responsible for reducing the amount of surface EGFR that can be targeted by EGFRab^{A647} (FIG. 17B). The ratio of EGFRab^{A647} that bound with EGFR^{GFP} was also found to be slightly increased upon the EGF treatment (FIG. 17C); however, no obvious difference in the ratio of EGFRab^{A647} interaction was found when endocytosis was suppressed (FIG. 17D). These results demonstrate that environmental alterations could affect the number of targetable molecules.

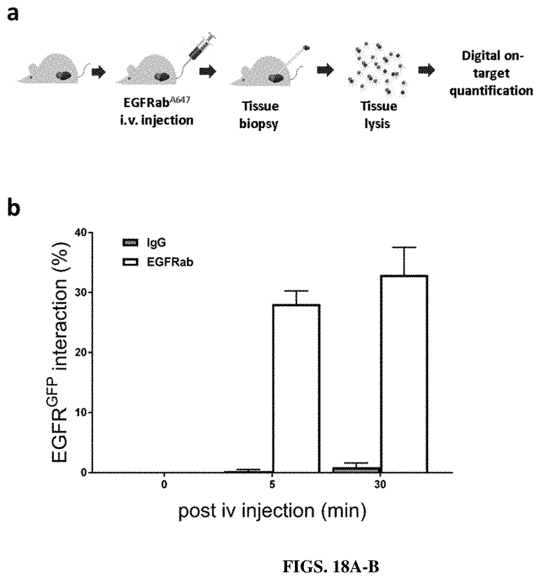

Next, the ability of DQON to detect antibody on-target interaction in vivo was evaluated. Because EGFRab has been used to treat colorectal cancer, a xenograft model of EGFR-expressing colorectal cancer was generated as previously described (Liao et al., 2015) (FIG. 18A). In brief, mice with subcutaneous tumors arising from colorectal cancer cells stably expressing EGFR^{GFP} (GEO-EGFR^{GFP} cells) received intravenous injections of EGFRab^{A647}, and small pieces of tumor tissue were collected at the indicated times. The tumor tissue samples were then degraded using tissue homogenizer with lysis buffer, centrifuged to remove large debris, and analyzed with DQON. Five minutes after injection, about 28% of the overall EGFR^{GFP} events were EGFRab^{A647}-EGFR^{GFP} events, and 30 minutes after injection, the ratio slightly increased to 33% (FIG. 18B). These results provide proof-of-concept evidence that DQON can directly detect and quantify on-target interaction in cells obtained from xenograft tumor models.

Antibody sensitivity and specificity, especially in living cells, are critical for targeted therapy, diagnosis, and research (Baker, 2015). The results provided herein show that DQON has the unique ability to directly detect and quantify cell-based antibody on-target interaction at the single-molecule level. Further, the interaction ratio of the target and antibody reveals the targeting efficacy. It was also demonstrated that the ratio of EGFR targeting is significantly reduced in the presence of EGF-induced endocytosis, which suggests that EGF neutralization or endocytosis suppression may increase the amount of EGFR to be targeted. This on-target interaction can also be evaluated in in vivo models, which mimic the treatment process. In summary, DQON as described herein provides a single molecule-based microfluidic platform to digitally quantify antibody on-target interaction. This platform can bridge the gap between in vitro and in vivo interaction assays and address the demand for drug on-target interaction evaluation. It can also be used in other applications, such as antibody validation and other interaction material analysis.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Baker, Nature, 521: 274-276, 2015.
Buter and Giaccone, Oncology, 19(13): 1707-1711, 2005.
Chemnitz et al., J Immunol. 173(2): 945-954, 2004.
Chosy et al., biotechnol Bioeng., 82:340-351, 2003.
Chou et al., Lab Chip,
Chou et al., Sci Signal, 7(315), 2014.
Chou et al., Curr Protoc Mol Biol., 114(1): 20.11.1-20.11.22, 2016.
Dang et al., Expert Rev Anticancer Ther., 16(1): 13-20, 2016.
Diggs and Hsueh, Biomark Res., 5: 12, 2017.
Enderlein et al., Curr Pharm Biotechno., 5(2): 155-161, 2004.
Engel and Kaklamani, Drugs, 67(9): 1329-1341, 2007.
Fedchenko and Reifenrath, Diagn Pathol., 9, 2014.
Friguet et al., J Immunol Methods, 77:305-319, 1985.
Frisk et al., Biosensors & Bioelectronics, 26(5): 1929-1935, 2011.
Govindan, Clin Cancer Res., 10:4241s-4244s, 2004.
Hahnefeld et al., Methods Mol Med., 94:299-320, 2004.
Harding and Burtness, Drugs Today (Barc), 41:107-127, 2005.
Hsu et al., Nat Cell Biol., 13(2): 174-181, 2011.
Hutchinson et al., J Transl Med., 13:217, 2015
Jafari and Hunger, Appl Immunohisto Mol Morph., 25(1): E12-E13, 2017.
Kameoka et al., Analytical Chemistry, 73:1935-1941, 2001.
Kim et al., Semin Oncol., 31:61-67, 2004.
Lee et al., Cell, 130(3): 440-455, 2007.
Lee et al., Am J Cancer Res., 5:2944-2958, 2015.
Levin and Carson, Differentiation, 72(1): 1-10, 2004.
Li et al., Nat Commun., 7, 2016.
Liao et al., J Clin Invest, 125: 4529-4543, 2015.
Maleki Vareki et al., Crit Rev Oncol Hematol., 116: 116-124, 2017.
Matos et al., Clinics (Sao Paulo), 61(5): 417-424, 2006.
Matos et al., Biomark Insights, 5: 9-20, 2010.
Peng et al., Chem Commun., 52(85): 12586-12589, 2016.
Phillips et al., Appl Immunohisto Mol Morph., 23(8): 541-549, 2015.
Ramos-Vara and Miller, Vet Pathol., 51(1): 42-87, 2014.
Shah et al., J Vis Exp., e51383, 2014.
Signore et al., Methods Mol Biol., 1606:51-70, 2017.
Slaughter and Li, Mol Biol Cell, 21(24): 4306-4311, 2010.
Spigel et al., J Clin Oncol., 31(32): 4105-4114, 2013.
Sweeney et al., Otolaryng Head Neck, 145:72, 2011.
Tekin and Gijs, Lab Chip, 13(24): 4711-4739, 2013.
Thompson et al., J Micromech Microeng., 20(11), 2010.
Trantum et al., Langmuir, 28(4): 2187-2193, 2012.
Wadhwa et al., J Immunol Methods, 382(1-2): 196-202, 2012.
Witters et al., Lab Chip, 13(11): 2047-2054, 2013.
Yaziji and Barry, Adv Anat Pathol., 13(5): 238-246, 2006.
Yeh et al., Nucleic Acids Res., 34(5): e35, 2006a.
Yeh et al., Nucleic Acids Res., 34(21): e144, 2006b.
Yu et al., Biosens Bioelectron, 41: 129-136, 2013.
Yuasa et al., Int J Clin Oncol. 22(4): 629-634, 2017.
Zhang et al., Nucleic Acids Res., 33(19): e166, 2005.
Zotova et al., J Immunol Methods, 439:8-14, 2016.

1. A method for determining the absolute concentration of an antigen of interest in a biological sample, comprising:
(a) providing a sample comprising an antigen of interest;
(b) contacting the biological sample with an antibody specific to the antigen of interest, wherein the antibody is conjugated to a detectable moiety;
(c) optionally applying the biological sample to a microfluidic chip comprising at least one microchannel; and
(d) digitally counting the detectable moiety as it passes a detection site, thereby obtaining the absolute concentration of the target protein.

2. The method of claim 1, wherein the applying comprises dispensing the sample onto a microchannel and applying electroosmotic force to the sample to move the solution through the microfluidic chamber.

3. The method of claim 1, wherein the method further comprises lysing the biological sample prior between steps (b) and (c).

4. The method of claim 3, wherein lysis comprises treatment with a lysis buffer.

5. The method of claim 4, wherein the lysis buffer comprises a detergent, a salt, and a buffering agent.

6. The method of claim 5, wherein the detergent is selected from group consisting of: non-ionic detergent, anionic detergent, cationic detergent, or zwitterionic detergent.

7. The method of claim 6, wherein the detergent is a nonionic detergent.

8. The method of claim 7, wherein the non-ionic detergent is NP-40.

9. The method of claim 7, wherein the non-ionic detergent is octyl-beta-glucoside.

10. The method of claim 7, wherein the non-ionic detergent is Triton X-100.

11. The method of claim 4, wherein lysis further comprises sonication.

12. The method of claim 1, wherein the detection is performed using a spectrophotometer, spectroscope, or confocal microscope.

13. The method of claim 12, further comprising optimizing the detection of the detectable moiety.

14. The method of claim 1, wherein the detectable moiety is a fluorophore.

15. The method of claim 14, wherein the fluorophores are selected from the group consisting of quantum dots, PE, PE-Cy5, PE-Cy7, APC, APC-Cy7, Qdot 565, qdot 605, Qdot 655, Qdot 705, green fluorescent protein (GFP), eGFP, TurboGFP, TagGFP2, mUKGEmerald GFP, Superfolder GFP, Azami Green, mWasabi, Clover, mClover3, mNeonGreen, NowGFP, Sapphire, T-Sapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSS-mOrange, CyOFP1, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP657, TagRFP675, mKate2, HcRed-Tandem, mPlum, mNeptune, NirFP, Kindling, far red fluorescent protein, yellow fluorescent protein (YFP), eYFP, TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKO_{K}, mKO2, mGrapel, mGrape2, zsYellow, eqFP611, Sirius, Sandercyanin, shBFP-N158S/L173I, near infrared proteins, iFP1.4, iRFP713, iRFP670, iRFP682, iRFP702, iRFP720, iFP2.0, mIFP, TDsmURFP, miRFP670, Brilliant Violet (BV) 421, BV 605, BV 510, BV 711, BV786, PerCP, PerCP/Cy5.5, Alexa Fluor dyes such as Alexa Fluor 350, 405, 430, 488, 514, 532, 546, 555, 568, 594, 633, 635, 647, 660, 680, 700, 750, and 790, FITC, BV570, BV650, DyLight 488, Dylight 649, and PE/Dazzle 594.

16. The method of claim 13, wherein optimizing comprises autofocusing the spectrophotometer, spectroscope, or microscope used to digitally detect the fluorophore.

17. The method of claim 16, wherein the spectrophotometer, spectroscope, or microscope is autofocused with a different wavelength laser than the laser used to excite the fluorophore.

18. The method of claim 16, wherein the spectrophotometer, spectroscope, or microscope is autofocused with a laser which excites the autofluorescence of the sample solution.

19. The method of claim 1, detecting comprises Fluorescence Correlation Spectroscopy (FCS) for the measurement of flow speed.

20.-65. (canceled)

66. A method for determining the on-target binding constant of a therapeutic agent to its target in a biological sample comprising:
(a) providing a therapeutic agent conjugated to a first detectable moiety and a biological sample comprising the target conjugated to a second detectable moiety;
(b) contacting the therapeutic agent with the biological sample to bind the therapeutic agent to its target in the biological sample;
(c) washing the biological sample to remove unbound therapeutic agent from the sample;
(d) optionally applying the biological sample to a microfluidic chip comprising at least one microchannel;
(e) digitally counting the first and second detectable moieties as they pass a detection site; and
(f) calculating the on-target binding constant from the digital count of detectable moieties.

67.-128. (canceled)
